(19) United States
(12) Patent Application Publication
(10) Pub. No.: US 2004/0086983 A1
(43) Pub. Date: May 6, 2004

(54) Method for producing a heterocyclic compound and an aromatic carboxylic acid having one or more hydroxyl groups, and modified aromatic ring dioxygenase

(76) Inventors: Norihiko Misawa, Kanagawa-ken (JP); Kazutoshi Shindo, Gunma-ken (JP); Hiroshi Okazaki, Tokyo-To (JP); Kensuke Furukawa, Fukuoka-ken (JP); Sueharu Horinouchi, Tokyo-To (JP)

Correspondence Address: FOLEY AND LARDNER, SUITE 500, 3000 K STREET NW, WASHINGTON, DC, 20007, US

(21) Appl. No.: 09/950,822

(22) Filed: Sep. 13, 2001

(30) Foreign Application Priority Data
Sep. 14, 2000 (JP) 2000-279703
Sep. 6, 2001 (JP) 2001-270878

(51) Int. Cl. C12P 17/10; C12P 17/12

(52) U.S. Cl. 435/121; 435/122

(57) ABSTRACT

An objective of the present invention is to provide a method of producing hydroxylated heterocyclic compounds and hydroxylated aromatic carboxylic acids by bioengineering technique, and modified enzymes which can be used for this method. A method of producing hydroxylated heterocyclic compounds or hydroxylated aromatic carboxylic acids comprises reacting an aromatic ring dioxygenase with heterocyclic compounds or aromatic carboxylic acids to hydroxylate these compounds. An enzyme according to the present invention is an aromatic ring dioxygenase comprising an α-subunit consisting of the amino acid sequence of SEQ ID NO: 2, which is modified according to the α-subunit of the biphenyl dioxygenase derived from the strain Burkholderia cepacia LB400, a β-subunit consisting of the amino acid sequence of SEQ ID NO: 4, and a ferredoxin consisting of the amino acid sequence of SEQ ID NO: 6, and a ferredoxin reductase consisting of the amino acid sequence of SEQ ID NO: 8.

### BACKGROUND OF THE INVENTION

1. Field of the Invention

The present invention relates to methods for producing heterocyclic compounds into which a hydroxyl group is introduced, and aromatic carboxylic acids into which a hydroxyl group is introduced, and novel modified enzymes which hydroxylate heterocyclic compounds and aromatic carboxylic acids. More specifically, the present invention relates to bioengineering transformation techniques for introducing a hydroxyl group into heterocyclic compounds or the like using recombinant microorganisms such as recombinant Escherichia coli or recombinant actinomycetes.

2. Background Art

In a method frequently used in today's research and development of pharmaceuticals, disease-associated molecule to be targeted by the manufactured drug (pharmaceutical target molecule) is first elucidated, after which high through-put screening (HTS) is carried out using this pharmaceutical target molecule with a certain biological activity as an index to search for hit compounds. In this method, a screening source library for a lead compound (generally, a lipoid compound having a molecular weight of about 100 to 700) to link to the drug for oral administration is necessary. The quality and quantity of this library are considered to be important in research and development of pharmaceuticals.

Today, many of the screening source libraries are chemically synthesized using techniques inorganic chemistry, such as combinatorial chemistry (Akihiro Tanaka, Drug Manufacturing and Combinatorial Chemistry, Protein, Nucleic Acid and Enzyme, 45, 887-894, 2000). Libraries derived from natural materials, such as microbial metabolites, are also used for research and development of drugs for oral administration. However, the use of the libraries derived from natural materials has been declining because of their high level of false positives, the time-consuming procedure for identification of active substances, and difficulty in finding novel compounds.

On the other hand, most of the chemically synthesized screening source libraries are non-specific. In a chemical synthesis, a bonding reaction, such as bonding of two precursors via —NHCO— bond, is easily carried out, but it is difficult to introduce a functional group, such as a hydroxyl group, into a specific site or stereospecifically into a compound. Furthermore, in the research and development of pharmaceuticals, after a lead compound which acts on a pharmaceutical target molecule is discovered using HTS, it is necessary to create analogs of the lead compound and determine the most appropriate candidate compound for development (lead optimization). At present, the analogs of the lead compound are also mainly produced by a chemical synthesis method, which means there will be the non-specificity of organic chemistry reactions.

Incidentally, virtually all oral drugs and synthetic dyes and over half of natural organic compounds contain heterocyclic groups. Therefore, a bioengineering conversion technique to specifically introduce functional groups, such as a hydroxyl group, into heterocyclic compounds is considered to be extremely important, and highly essential technique, in synthesizing building blocks, which are the starting structural units in the synthesis of oral drugs or drug-like compounds, as well as the chemical reactions to link to these and other compounds.

Furthermore, as the building block, a combination of an amine and a carboxylic acid, in particular, an amine having an aromatic ring in the molecule (hereinafter called an aromatic amine) and a carboxylic acid having an aromatic ring in the molecule (hereinafter called an aromatic carboxylic acid) is most frequently used. Therefore, a bioengineering conversion technique to specifically introduce a functional group, such as a hydroxyl group, into an aromatic amine and an aromatic carboxylic acid is also considered to be extremely important and highly essential technique.

The strain Pseudomonas pseudoalcaligenes KF707 is a polychlorinated biphenyl (PCB) decomposition bacterium isolated in Kita Kyushu by Kensuke Furukawa et al. of Department of Agriculture in Kyushu University. A gene encoding aromatic ring dioxygenase which is responsible for the first oxidation reaction of PCB was isolated from the strain P. pseudoalcaligenes KF707 and was named the biphenyl dioxygenase gene (bphA1A2A3A4 gene) (A. Suyama, R. Iwakiri, N. Kimura, A. Nishi, K. Nakamura, K. Furukawa, J. Bacteriol., 178, 4039-4046, 1996). The strain P. pseudoalcaligenes KF707 was grown in a medium containing biphenyl, 4-methylbiphenyl or diphenylmethane as a carbon source, but could not be grown in a medium containing benzene or toluene as a carbon source (A. Suyama, R. Iwakiri, N. Kimura, A. Nishi, K. Nakamura, K. Furukawa, J. Bacteriol., 178, 4039-4046, 1996). This difference can be attributed to a substrate specificity of the biphenyl dioxygenase which is involved in the first oxidation reaction.

The strain Burkholderia cepacia LB 400 (previously called Pseudomonas sp. LB 400) is a polychlorinated biphenyl decomposition bacteria isolated in New York State by Johnson et al. (Bedard, D. L., Unterman, R., Bopp, L. H., Brennan, M. J., Johnson, C., Appl. Environ. Microbiol., 51, 761-768, 1986). The strain B. cepacia LB 400 has been studied as a powerful PCB decomposition bacteria, along with the strain P. pseudoalcaligenes KF707, and its related genes and related enzymes have also been analyzed. Also from the strain B. cepacia LB400, a gene encoding aromatic ring dioxygenase which is responsible,for the first oxidation reaction of PCB was isolated and was named biphenyl dioxygenase gene (bphAEFG gene) (B. D. Erickson, E. J. MondeLB400o, J. Bacteriol., 174, 2903-2912, 1992).

The biphenyl dioxygenases (BDOs) of the strains Pseudomonas pseudoalcaligenes KF707 and B. cepacia LB400 showed extremely high homology at the amino acid sequence level. Namely, the degrees of homology were 94% for large subunits, 99% for small subunits, 100% for ferredoxin, and 100% for ferredoxin reductase. Nevertheless, they are different in substrate specificity and reaction specificity. For example, when 2,5,4′-trichlorobiphenyl was used as the substrate, BDO of the strain P. pseudoalcaligenes KF707 added oxygen atoms onto positions 2′ and 3′ of this substrate to produce cis-diol (see FIG. 1); while BDO of the strain B. cepacia LB400 added oxygen atoms onto position 3, and 4 to produce cis-diol (see FIG. 2) (N. Kimura, A. Nishi, M. Goto, K. Furukawa, J. Bacteriol., 179, 3936-3943, 1997). Furthermore, BDO of the strain KF707 was able to recognize diphenylmethane as a substrate for conversion while BDO of the LB400 strain was not able to recognize it as a substrate for conversion. On the other hand, 2,5,2′,5′-tetrachlorobiphenyl was recognized by BDO of the strain LB400 as a substrate for conversion (FIG. 2) while it was not recognized by BDO of the strain KF707 as a substrate for conversion.

Kensuke Furukawa et al. of Kyushu University isolated a DNA encoding a large subunit of the biphenyl dioxygenase derived from the strain LB400 and a large subunit of the biphenyl dioxygenase derived from the strain KF707 by PCR using a bphAI primer which comprises a common flanking sequence. Next, these DNAs were digested with DNase I, and 10 to 50 bp DNA fragments were recovered, mixed and subjected to self-priming PCR and PCR with the addition of. the bphA1 primer to yield various chimeric bphA1s in which amino acid sequences were randomly exchanged (DNA shuffling). It is reported that a variety of modified biphenyl dioxygenase genes (modified bphA1::bphA2AA3A4 genes) can be obtained by linking these chimera bphA1s upstream of three components (bphA2A3A4) other than the large subunit of the biphenyl dioxygenase derived from the strain P. pseudoalcaligenes KF707 (T. Kumamaru, H. Suenaga, M. Mitsuoka, T. Watanabe, K. Furukawa, Nature Biotechnology, 16, 663-666, 1998).

However, there are no reports to date that aromatic ring dioxygenases can introduce hydroxyl groups into heterocyclic compounds or aromatic carboxylic acids. Further, no enzyme that can specifically introduce hydroxyl groups into heterocyclic compounds or aromatic carboxylic acids has been reported.

### SUMMARY OF THE INVENTION

The present inventors have now found that a hydroxyl group can be introduced into compounds having a heterocyclic group in the molecule or into aromatic carboxylic acids using the biphenyl dioxygenase derived from the strain P. pseudoalcaligenes KF707, in a reaction-specific manner.

Also, the present inventors carried out DNA shuffling between the DNA encoding the large subunit of the biphenyl dioxygenase derived from the strain P. pseudoalcaligenes KF707 and the DNA encoding the large subunit of the biphenyl dioxygenase derived from the strain B. cepacia LB400, conducted the expression of a modified biphenyl dioxygenase gene comprising the DNA thus obtained and a DNA encoding the three components other than the large subunit, and found that a hydroxyl group can be introduced into compounds having a heterocyclic group in the molecule or aromatic carboxylic acids using the modified aromatic ring dioxygenase thus obtained, in a reaction-specific manner.

An objective of the present invention is to provide a method of producing hydroxylated heterocyclic compounds or hydroxylated aromatic carboxylic acids by bioengineering technique.

A method of producing hydroxylated heterocyclic compounds or hydroxylated aromatic carboxylic acids according to the present invention comprises the step of reacting an aromatic ring dioxygenase with heterocyclic compounds or aromatic carboxylic acids to hydroxylate these compounds.

According to the method of the present invention, hydroxylated heterocyclic compounds and hydroxylated aromatic carboxylic acids can be easily produced at low costs.

Another objective of the present invention is to provide a modified enzyme which can efficiently hydroxylate heterocyclic compounds or aromatic carboxylic acids.

A modified enzyme of the present invention is a modified aromatic ring dioxygenase comprising a tetramer consisting of

an α-subunit consisting of a modified amino acid sequence of SEQ ID NO: 2 which has one or more modifications selected from the group consisting of a substitution, a deletion, an insertion and an addition, and has been modified according to the amino acid sequence of the α-subunit of the biphenyl dioxygenase derived from the strain Burkholderia cepacia LB400;

a β-subunit consisting of the amino acid sequence of SEQ ID NO: 4 or a modified amino acid sequence of SEQ ID NO: 4 having one or more modifications selected from the group consisting of a substitution, a deletion, an insertion and an addition;

a ferredoxin consisting of the amino acid sequence of SEQ ID NO: 6 or a modified amino acid sequence of SEQ ID NO: 6 having one or more modifications selected from the group consisting of a substitution, a deletion, an insertion and an addition; and

a ferredoxin reductase consisting of the amino acid sequence of SEQ ID NO: 8 or a modified amino acid sequence of SEQ ID NO: 8 having one or more modifications selected from the group consisting of a substitution, a deletion, an insertion and an addition.

Still another objective of the present invention is to provide an α-subunit of an aromatic ring dioxygenase which is modified to efficiently hydroxylate heterocyclic compounds and aromatic carboxylic acids.

A modified α-subunit of an aromatic ring dioxygenase according to the present invention consists of a modified amino acid sequence of SEQ ID NO: 2 which has one or more modifications selected from the group consisting of a substitution, a deletion, an insertion and an addition, which has been modified according to the amino acid sequence of the α-subunit of the biphenyl dioxygenase derived from the strain Burkholderia cepacia LB400.

### BRIEF DESCRIPTION OF THE DRAWINGS

FIG. 1 shows substrates recognized by the biphenyl dioxygenase derived from the strain Pseudomonas pseudoalcaligenes KF707, and reaction products thereof.

FIG. 2 shows substrates recognized by the biphenyl dioxygenase derived from the strain Burkholderia cepacia LB400, and the reaction products thereof.

FIG. 3 shows the aligned amino acid sequences of the α-subunit (KF707) derived from the strain Pseudomonas pseudoalcaligenes KF707, the α-subunit (LB400) derived from the strain Burkholderia cepacia LB400, and a modified α-subunit (2072) derived from the strain Pseudomonas pseudoalcaligenes KF707.

FIG. 4 shows chemical structures of heterocyclic compounds which can be used as a substrate in the present invention.

FIG. 5 shows chemical structures of heterocyclic compounds which can be used as a substrate in the present invention.

FIG. 6 shows chemical structures of heterocyclic compounds which can be obtained as a converted product in the present invention.

FIG. 7 shows chemical structures of heterocyclic compounds which can be obtained as a converted product in the present invention.

FIG. 8 shows chemical structures of flavonoid, phthalimide derivatives having aromatic rings, and aromatic carboxylic acids, which can be used as a substrate in the present invention.

FIG. 9 shows chemical structures of flavonoids, phthalimide derivatives having aromatic rings, and aromatic carboxylic acids, which can be obtained as a converted product in the present invention.

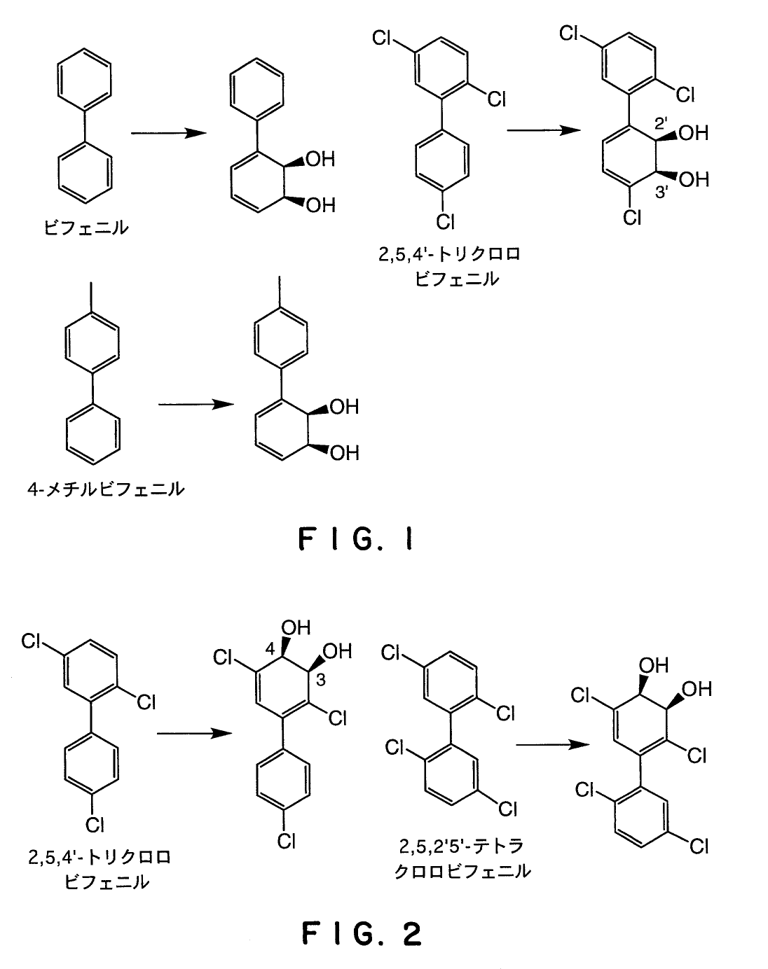

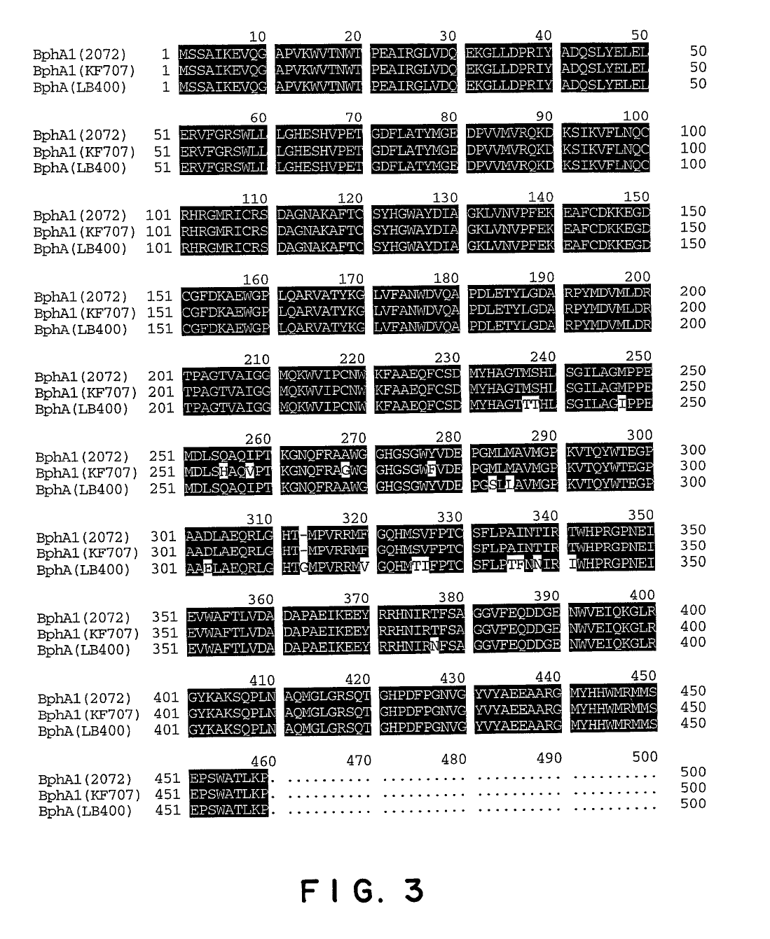

### DETAILED DESCRIPTION OF THE INVENTION

Deposition of Microorganisms

The strain of Escherichia coli JM 109 (pKF6622) into which the (aromatic ring dioxygenase gene derived from the strain Pseudomonas pseudoalcaligenes KF707)is incorporated was deposited at the National Institute of Bioscience and Human-Technology Agency of Industrial Science and Technology, the Ministry of International Trade and Industry (1-3 Higashi 1-Chome, Tsukuba City, Ibaraki Prefecture, Japan) on Sep. 13, 2000. The accession number is FERM BP-7300.

The strain of Escherichia coli JM 109 (pKF2072) into which the modified aromatic ring dioxygenase gene is incorporated was deposited at the National Institute of Bioscience and Human-Technology Agency of Industrial Science and Technology, the Ministry of International Trade and Industry (1-3 Higashi 1-Chome, Tsukuba City, Ibaraki Prefecture, Japan) on Sep. 13, 2000. The accession number is FERM BP-7299.

Aromatic Ring Dioxygenase and Gene Thereof

The term “aromatic ring dioxygenase” refers to an enzyme which acts on an aromatic ring such as a benzene ring and introduces a diatomic oxygen molecule into the aromatic ring. As a result of the introduction of the diatomic oxygen molecule into the aromatic ring, two hydroxyl groups are introduced into the aromatic ring.

The aromatic ring dioxygenase can consist of four subunits, i.e., a tetramer, consisting of an aromatic ring dioxygenase large subunit (α-subunit) (BphA1), an aromatic ring dioxygenase small subunit (β-subunit) (BphA2), a ferredoxin (BphA3), and a ferredoxin reductase (known as NAD(P)H-ferredoxin reductase) (BphA4).

In the present invention, the aromatic ring dioxygenase can be (1) an aromatic ring dioxygenase derived from Pseudomonas pseudoalcaligenes and its modified form still having aromatic ring dioxygenase activity, and (2) a modified aromatic ring dioxygenase derived from Pseudomonas pseudoalcaligenes, of which α-subunit has been modified according to the biphenyl dioxygenase derived from the strain Burkholderia cepacia LB400 (modified BphA1-BphA2-BphA3BphA4).

(1) Aromatic Ring Dioxygenase Derived from Pseudomonas pseudoalcaligenes and Modified Form Thereof

The aromatic ring dioxygenase can be a biphenyl dioxygenase derived from Pseudomonas pseudoalcaligenes, in particular, the biphenyl dioxygenase derived from the strain Pseudomonas pseudoalcaligenes KF707 (BphA1-BphA2-BphA3BphA4) (A. Suyama, R. Iwakiri, K. Kimura, A. Nishi, K. Nakamura, K. Furukawa, J. Bacteriol., 178, 4039-4046, 1966).

The amino acid sequences of the α-subunit, β-subunit, ferredoxin, and ferredoxin reductase of the biphenyl dioxygenase derived from the strain Pseudomonas pseudoalcaligenes KF707 can be the amino acid sequences of SEQ ID NOs: 2, 4, 6, and 8, respectively.

In the present invention, the amino acid sequences of SEQ ID NOs: 2, 4, 6, and 8 may have one or more modifications, for example, one to several modifications, selected from the group consisting of a substitution, a deletion, an insertion, and an addition. In this case, the tetramer consisting of an α-subunit consisting of the amino acid sequence of SEQ ID NO: 2 which may be modified, a β-subunit consisting of the amino acid sequence of SEQ ID NO: 4 which may be modified, a ferredoxin consisting of an amino acid sequence of SEQ ID NO: 6 which may be modified, and a ferredoxin reductase consisting of the amino acid sequence of SEQ ID NO: 4 which may be modified has the aromatic ring dioxygenase activity.

In the present invention, the aromatic ring dioxygenase which is characterized in that

the α-subunit consisting of the amino acid sequence of SEQ ID NO: 2 or an amino acid sequence having 80% or more, preferably 90% or more, more preferably 95% or more homology to the amino acid sequence of SEQ ID NO: 2,

the β-subunit consisting of the amino acid sequence of SEQ ID NO: 4 or an amino acid sequence having 80% or more, preferably 90% or more, more preferably 95% or more homology to the amino acid sequence of SEQ ID NO: 4,

the ferredoxin consisting of the amino acid sequence of SEQ ID NO: 6 or an amino acid sequence having 80% or more, preferably 90% or more, more preferably 95% or more homology to the amino acid sequence of SEQ ID NO: 6,

the ferredoxin reductase consisting of the amino acid sequence of SEQ ID NO: 8 or an amino acid sequence having 80% or more, preferably 90% or more, more preferably 95% or more homology to the amino acid sequence of SEQ ID NO: 8, and

the tetramer consisting of the α-subunit, β-subunit, ferredoxin, and ferredoxin reductase has the aromatic ring dioxygenase activity can be used for the hydroxylation of heterocyclic compounds and aromatic carboxylic acids.

In the present specification, whether or not to “have aromatic ring dioxygenase activity” can be evaluated by reacting the protein of interest with a substrate and detecting whether or not the substrate conversion reaction occurs. For example, whether or not to “have aromatic ring dioxygenase activity” can be evaluated according to the methods described in Examples 4 and 5.

(2) Modified Aromatic Ring Dioxygenase (Modified BphA1-BphA2-BphA3-BphA4)

An aromatic ring dioxygenase according to the present invention can be a biphenyl dioxygenase derived from Pseudomonas pseudoalcaligenes, in particular, the biphenyl dioxygenase derived from the strain Pseudomonas pseudoalcaligenes KF707, in which the α-subunit is optimized according to the α-subunit of the biphenyl dioxygenase derived from the strain Burkholderia cepacia LB400 (modified BphA1-BphA2-BphA3-BphA4).

Accordingly, the α-subunit of the biphenyl dioxygenase derived from the strain Pseudomonas pseudoalcaligenes KF707 can consist of a modified amino acid sequence of SEQ ID NO: 2 which has one or more modifications selected from the group of a substitution, a deletion, an insertion, and an addition, and has been modified according to the amino acid sequence of the α-subunit of the biphenyl dioxygenase derived from the strain Burkholderia cepacia LB400. The amino acid sequences of the β-subunit, ferredoxin, and ferredoxin reductase of the biphenyl dioxygenase derived from the strain Pseudomonas pseudoalcaligenes KF707 can be the amino acid sequences of SEQ ID NOs: 4, 6 and 8, which may be modified, respectively. In this case, the four subunits consisting of the α-subunit consisting of the amino acid sequence of SEQ ID NO: 2 which has been modified according to the amino acid sequence of the α-subunit derived from the strain Burkholderia cepacia LB400, the β-subunit consisting of the amino acid sequence of SEQ ID NO: 4 which may be modified, the ferredoxin consisting of the amino acid sequence of SEQ ID NO: 6 which may be modified, and the ferredoxin reductase consisting of the amino acid sequence of SEQ ID NO: 8 which may be modified has the aromatic ring dioxygenase activity.

The amino acid sequence of the α-subunit derived from the strain Burkholderia cepacia LB400 can be the amino acid sequence of SEQ ID NO: 11. The nucleotide sequence of the biphenyl dioxygenase gene derived from the strain Burkholderia cepacia LB400, namely the bphAEFG gene, is registered at GenBank under the accession number M86348.

In the present invention, the expression “be modified according to the amino acid sequence of the α-subunit derived from the strain Burkholderia cepacia LB400” means that the amino acid sequence of the α-subunit derived from the strain Pseudomonas pseudoalcaligenes KF707 are compared with the amino acid sequence of the α-subunit derived from the strain Burkholderia cepacia LB400 and then that one or more amino acid residues of the α-subunit derived from the strain Pseudomonas pseudoalcaligenes KF707, which are different from the amino acid residues of the α-subunit derived from the strain Burkholderia cepacia LB400, are substituted with the corresponding amino acid residues of the α-subunit derived from the strain Burkholderia cepacia LB400. If an amino acid residue of the α-subunit derived from the strain Pseudomonas pseudoalcaligenes KF707 has no corresponding amino acid residue in the α-subunit derived from Burkholderia cepacia LB400, the amino acid residue of the α-subunit derived from the strain Pseudomonas pseudoalcaligenes KF707 can be deleted. If an amino acid residue of the α-subunit derived from the strain Burkholderia cepacia LB400 has no corresponding amino acid residue in the α-subunit derived from Pseudomonas pseudoalcaligenes KF707, the corresponding amino acid residue of the α-subunit derived from the strain Burkholderia cepacia LB400 can be inserted into the α-subunit derived from the strain Pseudomonas pseudoalcaligenes KF707.

The amino acid sequence of SEQ ID NO: 10 is an example of the amino acid of SEQ ID NO: 2 modified according to the amino acid sequence of the α-subunit derived from the strain Burkholderia cepacia LB400.

For example, optimization of the aromatic ring dioxygenase can be carried out as follows.

A DNA encoding the large subunit of the biphenyl dioxygenase derived from the strain Burkholderia cepacia LB400 and a DNA encoding the large subunit of the biphenyl dioxygenase derived from the strain Pseudomonas pseudoalcaligenes KF707 are isolated by PCR using a bphA1 primer comprising a common flanking sequence. Next, these DNAs are digested with DNase I, 10 to 50 bp DNA fragments are recovered and mixed, and then self-priming PCR, or PCR using the bphA1 primer are carried out to obtain a variety of chimeric bphA1s in which amino acid sequences are randomly exchanged (DNA shuffling) (see Example 1).

The chimeric bph1s thus obtained are each linked to an expression vector along with the bphA2A3A4 to measure the substrate conversion reaction. An aromatic ring dioxygenase produces a meta-cleavage product when it acts on a substrate. Since meta-cleavage products generally yield a yellow color, they can be monitored at 434 nm. Next, the ability to convert various aromatic hydrocarbons (activity to introduce hydroxyl groups) was examined using the transformants. Optimized amino acid sequences and nucleotide sequences can be obtained by selecting transformants using the ability to convert aromatic hydrocarbons as an index, and then analyzing incorporated genes according a conventional method.

Among the transformants carrying optimized genes, the E. coli pKF2072 transformant was found to have an extremely broad substrate specificity. The base sequence of the large subunit gene in the modified biphenyl dioxygenase gene (modified bphA1) contained in this plasmid pKF2072 and the amino acid sequence encoded by this base sequence are shown in SEQ ID NOs: 9 and 10, respectively. Occasionally, this modified α-subunit is called BphA1 (2072) and this gene is called bphA1 (2072). The BphA1 (2072) was different from the large subunit of the biphenyl dioxygenase derived from its parent strain Pseudomonas pseudoalcaligenes KF707 (occasionally called BphA1 (KF707)) in 4 amino acids, and was different from the large subunit of the biphenyl dioxygenase derived from the other parent strain Burkholderia cepacia LB400 (occasionally called BphA (LB400)) in 15 amino acids. A comparison of the amino acid sequences of these three large subunits is shown in FIG. 3.

An embodiment of the present invention provides a method for producing hydroxylated heterocyclic compounds or aromatic carboxylic acids, which comprises the step of reacting a culture medium obtained by culturing microorganisms transformed to express an aromatic ring dioxygenase gene with heterocyclic compounds or aromatic carboxylic acids, and hydroxylating the heterocyclic compounds or aromatic carboxylic acids. In this case, the transformants are first cultured and then the resulting culture medium is brought into contact with heterocyclic compounds or aromatic carboxylic acids to hydroxylate these compounds, or alternatively, the transformants can be cultured in a medium containing heterocyclic compounds or aromatic carboxylic acids. Further in a method of the present invention, the trans formants can be present or absent, as long as the enzyme produced from the transformants functions.

The aromatic ring dioxygenase gene can be a DNA encoding the aromatic ring dioxygenase.

As mentioned above, the aromatic ring dioxygenase includes (1) an aromatic ring dioxygenase derived from Pseudomonas pseudoalcaligenes and its modified form still having aromatic ring dioxygenase activity, and (2) a modified aromatic ring dioxygenase derived from Pseudomonas pseudoalcaligenes, of which α-subunit is modified according to the biphenyl dioxygenase derived from the strain Burkholderia cepacia LB400 (modified BphA1-BphA2-BphA3BphA4). Given an amino acid sequence of the aromatic ring dioxygenase, a nucleotide sequence encoding it can be readily determined. Namely, nucleotide sequences encoding the amino acid sequences of SEQ ID NOs: 2, 4, 6, 8, and 10 and modified sequences thereof can be selected. Accordingly, DNA sequences encoding an aromatic ring dioxygenase consisting of four subunits include a part or all of the DNA sequences of SEQ ID NOs: 1, 3, 5, 7, and 9, as well as DNA sequences encoding the same amino acid and having degenerate codons, and further include RNA sequences corresponding to these DNA sequences.

In the present invention, an aromatic ring dioxygenase gene can be used for the hydroxylation of heterocyclic compounds and aromatic carboxylic acids, said aromatic ring dioxygenase gene is characterized in that

a DNA sequence encoding the α-subunit is the DNA sequence of SEQ ID NO: 1 or a DNA sequence having 80% or more, preferably 90% or more, more preferably 95% or more homology to the DNA sequence of SEQ ID NO: 1,

a DNA sequence encoding the β-subunit is the DNA sequence of SEQ ID NO: 3 or a DNA sequence having 80% or more, preferably 90% or more, more preferably 95% or more homology to the DNA sequence of SEQ ID NO: 3,

a DNA sequence encoding the ferredoxin is the DNA sequence of SEQ ID NO: 5 or a DNA sequence having 80% or more, preferably 90% or more, more preferably 95% or more homology to the DNA sequence of SEQ ID NO: 5,

a DNA sequence encoding the ferredoxin reductase is the DNA sequence of SEQ ID NO: 7 or a DNA sequence having 80% or more, preferably 90% or more, more preferably 95% or more homology to the DNA sequence of SEQ ID NO: 7, and

the tetramer consisting of the α-subunit, β-subunit, ferredoxin, and ferredoxin reductase, which are encoded by these DNA sequences, has the aromatic ring dioxygenase activity.

DNA sequences encoding the α-subunit, β-subunit, ferredoxin, and ferredoxin reductase of the aromatic ring dioxygenase derived from the strain Pseudomonas pseudoalcaligenes KF707can be SEQ ID NOs: 1, 3, 5, and 7, respectively. The base sequence of the biphenyl dioxygenase gene derived from the strain Burkholderia cepacia LB400, namely the bphA1A2A3A4 gene, is registered at GenBank under the accession number M83673.

An example of the DNA sequence encoding a modified amino acid sequence of SEQ ID NO: 2 according to the amino acid sequence of the α-subunit derived from the strain Burkholderia cepacia LB400 is the nucleotide sequence of SEQ ID NO: 9.

Gene Introduction and Gene Expression

Microorganisms to be trans formed to express an aromatic ring dioxygenase gene can be those transformed with an expression vector to which the aromatic ring dioxygenase gene is linked.

In the present invention, the construction of the expression vector and the introduction of the expression vector into a host and its expression can be carried out according to the procedures and methods conventionally used in the field of genetic engineering. For example, for the construction of plasmids, and the introduction and expression of the plasmids, see “Vectors for Cloning Genes,” Methods in Enzymology, 216, pp. 469-631, 1992, Academic Press; “Other Bacterial Systems,” Methods in Enzymology, 204, pp. 305-636, 1991, Academic Press; and “A Laboratory Manual for Gene Expression,” edited by Isao Ishida and Tamio Ando, 1994, Kodansha. For the selection of transformants and the culture conditions, see, for example, Sambrook, J., Fritsch, E. F., Maniatis, T., “Molecular Cloning—A Laboratory Manual,” Cold Spring Harbor Laboratory Press, 1989 or List of Cultures 10th Edition, by Institute for Fermentation, Osaka, 1996.

The expression vector of the present invention can be constructed, for example, by operably linking a promoter upstream of an aromatic ring dioxygenase gene and a terminator downstream of an aromatic ring dioxygenase gene and, optionally, by operably linking a gene marker and/or other regulatory sequences. The linkage of the promoter and the terminator to the gene according to the present invention, and the insertion of the expression unit into the vector can be carried out according to a conventional method.

Microorganisms transformed to express an aromatic ring dioxygenase gene can be those to which the aromatic ring dioxygenase gene as such is directly introduced. The direct introduction of the gene to a host can be carried out according to a conventional method.

The introduction of foreign genes into representative microorganisms usable in the present invention and the expression of these genes are briefly explained as follows.

(1) Escherichia coli

The introduction of a foreign gene into Escherichia coli can be carried out using established, effective methods, such as the procedure of Hanahan or the rubidium method (see, for example, J. Sambrook, E. F. Fritsch, T. Maniatis, “Molecular Cloning—A Laboratory Manual,” Cold Springs Harbor Laboratory Press, 1989). The expression of a foreign gene in E. coli can be carried out according to a conventional method (see, for example, “Molecular Cloning—A Laboratory Manual,” and “A Laboratory Manual for Gene Expression,” Kodansha). Also, the expression can be carried out, for example, using a vector for E. coli carrying a lac promoter of the pUC system, the pBluescript system, or the like, or a T7 promoter such as pT7-7

(2) Actinomycetes

The host-vector system has been established for several actinomycetes such as Streptomyces lividans. For example, an expression vector pIJ6021 has the kanamycin (Km) resistant gene as a drug-resistant marker gene, which can be induced with thiostrepton (see E. Takano, J. White, C. J. Thompson, M. J. Bibb, Gene, 166, 133-137, 1995).

(3) Yeasts

The introduction of a foreign gene into yeast Sacchromyces cerevisiae can be carried out using established, effective methods such as the lithium method (see, for example, “New Biotechnology in Yeast,” edited by Yuichi Akiyama, compiled by Bioindustry Association, Igaku Shuppan Center). The expression of a foreign gene in yeast can be carried out by constructing an expression cassette using a promoter and a terminator such as PGK and GPD, to which the foreign gene is inserted between the promoter and the terminator to allow transcriptional read-through to occur, and by inserting this expression cassette into a vector for S. cerevisiae, such as a YEp system vector (a multicopy vector for yeast having a ARS sequence of the yeast chromosome as the replication origin), a YEp system vector (a multicopy vector for yeast having the replication origin of the 2 μm DNA of yeast), and a YIp system vector (a vector for integrating a yeast chromosome having no replication origin of yeast) (see “New Biotechnology in Yeast,” Igaku Shuppan Center; Nippon Nogei-Kagaku Kai ABC Series “Genetic Engineering for Producing Materials,” Asakura Shoten Co., Ltd.; and Yamano, S., Ishii, T., Nakagawa, M., Ikenaga, H., and Misawa, N., “Metabolic Engineering for Production of β-Carotene and Lycopene in Sacchromyces cerevisiae,” Biosci. Biotech. Biochem., 58, 1112-1114, 1994).

A foreign gene can be introduced into yeast Candida utilis according to the method described in Japanese Patent Application Laid-open Publication No. 173170/1996. More specifically, a drug resistant marker gene such as a cycloheximide resistant gene, a G418 resistant gene, and a hygromycin resistant gene is made into a linier chain, and then incorporated into a chromosome by the electric pulse method or the lithium method. A promoter such as GAP and PGK described in Japanese Patent Application Laid-open Publication No. 173170/1996 can be used for the expression of the foreign gene.

Cultivation of Transformants and Conversion Reaction of Substrates

Transformed microorganisms can be cultured using an ordinary culture method. On cultivation, an appropriate selective stress such as the addition of antibiotics can be used so that deletion of a vector carrying a foreign gene, i.e., an aromatic ring dioxygenase gene, can be prevented. Peptones, yeast extract, saccharides and organic substances can be used as a medium. A liquid culture method is most appropriately used. A culture temperature is preferably 16° C. to 40° C., in particular 20° C. to 30° C. A medium pH during cultivation is preferably maintained at pH 4 to 10, in particular at pH 6 to 8. Further, it is desirable to induce gene expression in order to produce a large amount of aromatic ring dioxygenase in the cells. For example, recombinant E. coli cells can be induced with IPTG after the culture reaches an optical density (OD 600 nm) of about 1. Further, converted products can be accumulated in the medium or cells by co-cultivation with the addition of a substrate generally for ½ to 4 days. The degree of converted can be determined by an HPLC analysis.

Various methods are applicable for the HPLC analysis of the resulting products. However, it is preferable to use a C18 column with a gradient in order to efficiently isolate various heterocyclic compounds and their products in a single column. Further, a photodiode array detector is preferably used to efficiently perform ultraviolet absorption spectrum analysis of the peak.

For example, the cultivation and conversion reaction using E. coli cells as a host can be carried out as follows.

Generally, many microorganisms including E. coli can be stored almost indefinitely by suspending cells in 15 to 50% glycerol and storing them at −70° C. to −80° C. in a deep freezer (glycerol stock). Accordingly, transformants can be stored as glycerol stock cells.

Purification and Identification of Products

Products can be purified using a suitable method generally used for small organic compounds.

Products can be purified based on the principle of extraction. For example, products in a culture filtrate can be extracted with a water immiscible organic solvent such as ethyl acetate, or products in the cells can be recovered by treating the cells obtained by filtration or centrifugation with methanol, ethanol, acetone or the like. The culture can be subjected to the abovementioned extraction process without isolating the cells. Another extraction method can be a countercurrent distribution method using an appropriate solvent.

Also, products can be purified based on the principle of adsorption. A product-containing fluid, such as a culture filtrate and an extract obtained by the abovementioned extraction procedure, can be treated with an appropriate adsorbent, such as silica gel, activated carbon, and “Dia Ion HP-20” (Mitsubishi Kasei, Corp.) to adsorb the product of interest, and then the adsorbed product can be eluted with an appropriate solvent to obtain the product. The product solution thus obtained is concentrated and dried under reduced pressure to obtain a crude product material.

The crude product material thus obtained can be further purified by carrying out the abovementioned extraction method and adsorption method, if necessary, in combination with high performance liquid chromatography or the like, repeatedly as necessary. For example, column chromatography using an adsorbent such as silica gel or a gel filtration agent such as “Sephadex LH-20” (Pharmacia), high performance liquid chromatography using UYMC Pack“(Yamamura Kagaku), and a countercurrent distribution method can be carried out in combination.

The product can be identified by ¹H-NMR and ¹³C—NMR spectrum analyses, an MS spectrum analysis, or the like.

Heterocyclic Compounds and Hydroxylated Heterocyclic Compounds

In the present specification, the term “heterocyclic compound” refers to a compound having a heterocyclic group in the molecule.

In the present specification, the term “heterocyclic group” refers to a monocyclic or bicyclic group comprising one or more heteroatoms selected from the group consisting of a nitrogen atom, oxygen atom, and sulfur atom, and said group may be substituted with a substitute group.

Examples of the “heterocyclic group” include a 5- to 7-membered saturated or unsaturated monocyclic heterocyclic group which may be substituted with a C₁₋₄ alkyl group, and a 9- to 11-membered saturated or unsaturated bicyclic heterocyclic group which may be substituted with a C₁₋₄ alkyl group.

Examples of heterocyclic rings comprising the “heterocyclic group” include quinoline, indole, indanone, benzothiazole, benzoxazole, pyridine, 3-methylpyridine, pyrimidine, pyrrole, pyrazole, 3-methylpyrazole, imidazole, isothiazole, benzofuran, thiophene, chromone (4H-chromene-4-on), chroman-4-on, 6-hydroxy-chroman-4-on, and phthalimide.

When the heterocyclic group of a heterocyclic compound is benzoxazole, the heterocyclic group in the hydroxylated heterocyclic group can be cis-4,5-dihydrobenzoxazolediol.

When the heterocyclic group of a heterocyclic compound is indole, the heterocyclic group in the hydroxylated heterocyclic group can be 5-hydroxyindole.

When the heterocyclic group of a heterocyclic compound is pyrazole, the heterocyclic group in the hydroxylated heterocyclic group can be 4-hydroxypyrazole.

When the heterocyclic group of a heterocyclic compound is pyridine, the heterocyclic group in the hydroxylated heterocyclic group can be 3-hydroxypyridine.

When the heterocyclic group of a heterocyclic compound is benzofuran, the heterocyclic group in the hydroxylated heterocyclic group can be 5-hydroxybenzofuran or 6-hydroxybenzofuran.

When the heterocyclic group of a heterocyclic compound is thiophene, the heterocyclic group in the hydroxylated heterocyclic group can be 2,3-dihydroxy-2,3-dihydrothiophene.

A heterocyclic compound may have an unsubstituted phenyl group in addition to a heterocyclic group. More specifically, it can be represented by the formula (I):

Het-Alkyl-R¹ (I)

wherein Het is a heterocyclic group, Alkyl is a bond or an optionally-branched alkylene chain having 1 to 4 carbon atoms, and R¹ is an unsubstituted phenyl group).

When the heterocyclic compound is a compound of the formula (I), the hydroxylated heterocyclic compound can be represented by the formula (I′):

Het-Alkyl-R^{1′} (I′)

wherein Het and Alkyl are the same as defined in the formula (I), and R^{1′} is any one of the following groups:

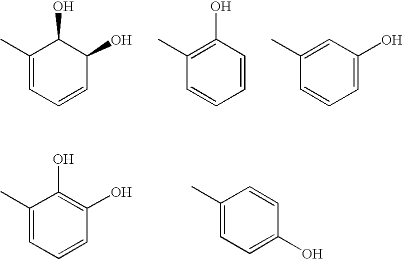

In the formula (I) and formula (I′), Alkyl is preferably —(CH₂)ₙ— (in which n is an integer of 0 to 4).

If Alkyl represents a bond, or n=0, the heterocyclic compound is a heterocyclic phenyl, or a compound in which a heterocyclic group and a phenyl group are single-bonded. When the substrate is a “heterocyclic phenyl”, a heterocyclic group-cis-2,3-dihydrobenzenediol (a heterocyclic group-cis-2,3-dihydroxycyclohexa-4,6-diene, in which the positions 2 and 3 of the phenyl group form cis-diol) can be obtained as the reaction product by a stereospecific reaction. When the substrate is a “heterocyclic phenyl”, one hydroxyl group can also be introduced into the position 2 of the phenyl group. In this case, examples of the substrate include 2-phenylindole and 3-methyl-1-phenylpyrazole.

When Alkyl is methylene, or n=1, the heterocyclic compound is a heterocyclic benzyl, or a compound in which a heterocyclic group and a phenyl group are bonded via methylene. When the substrate is a “heterocyclic benzyl”, a heterocyclic group-methylene-cis-2,3-dihydrobenzenediol (a heterocyclic group-methylene-cis-2,3dihydroxycyclohexa-4,6-diene, in which the positions 2 and 3 of the phenyl group form cis-diol) can be obtained as the reaction product by a stereospecific reaction. When the substrate is a “heterocyclic benzyl”, one hydroxyl group can also be introduced into the position 2 of the phenyl group. In this case, an example of the substrate is 4-benzyl isothiazole.

When Het in the formula (I) is indole, the hydroxylated heterocyclic compound can be a compound of the formula (I) in which Het is 5-hydroxyindole.

When Het in the formula (I) is pyrazole, the hydroxylated heterocyclic compound can be a compound of the formula (I) in which Het is 4-hydroxypyrazole.

In the production of pharmaceuticals and chemical compounds, organic synthetic reactions in which cis-diols are used as a building block are known (for example, see T. Hudlicky, A. J. Thorpe, Chem. Commun., 1993-2000, 1996; D. R. Boyd, G. N. Sheldrake, Natural Product Report, 309-324, 1998; or T. Hudlicky, D. Gonzalez, D. T. Gibson, Aldrichimia Acta, Vol. 32, Number 2, 35-62, 1999). Accordingly, the resulting cis-diol derivatives are useful for manufacturing building blocks in the chemical syntheses to link to the pharmaceuticals and other chemical compounds.

A heterocyclic compound may. have a substituted phenyl group in addition to a heterocyclic group. More specifically, it can be represented by the formula (II):

Het-Alkyl-R² (II)

wherein Het is a heterocyclic group, Alkyl is a bond or an optionally-branched alkylene chain having 1 to 4 carbon atoms, and R² is a phenyl group substituted with a C₁₋₄ alkyl group or a hydroxyl group.

In the formula (II), Het can be benzoxazole or pyridine, and R² can be 2-hydroxyphenyl or 4-methylphenyl.

When the heterocyclic compound is a compound of the formula (II), the hydroxylated heterocyclic compound can be represented by the formula (II′):

Het′-Alkyl-R² (II′)

wherein R² and Alkyl are the same as defined in the formula (II), and Het, is a heterocyclic group substituted with 1 or 2 hydroxyl groups. A compound of the formula (II′) is characterized in that the hydroxyl groups are introduced into the heterocyclic group.

In the formula (II) and the formula (II′), Alkyl is preferably —(CH₂)ₚ— (in which p is an integer of 0 to 4).

In the formula (II), when Het is benzoxazole and R² is 2-hydroxyphenyl, Het′ in the formula (II′) can be 4,5dihydroxy-4,5-dihydrobenzoxazole.

In the formula (II), when Het is pyridine and R² is 4-methylphenyl, Het′ in the formula (II′) can be 3hydroxypyridine.

Further, a heterocyclic compound may have a hydrocarbon chain in addition to a heterocyclic group. More specifically, it can be represented by the formula (III):

Het-Alkyl-H (III)

wherein Het is a heterocyclic group, Alkyl is an optionally-branched alkylene chain having 1 to 8 carbon atoms.

Het can be benzofuran or thiophene.

When the heterocyclic compound is a compound of the formula (III), the hydroxylated heterocyclic compound can be represented by the formula (III′):

Het′-Alkyl-H (III′)

wherein Het′ is a heterocyclic group substituted with 1 or 2 hydroxyl groups and Alkyl is the same as defined in the formula (III). A compound of the formula (III′) is characterized in that the hydroxyl groups are introduced into the heterocyclic group.

In the formula (III) and the formula (III′), Alkyl is preferably —(CH₂)ᵣ— (in which r is an integral of 1 to 8).

In the formula (III), when Het is benzofuran, Het′ in the formula (III′) can be 3-hydroxybenzofuran or 4-hydroxybenzofuran.

In the formula (III), when Het is thiophene, Het′ in the formula (III′) can be 2,3-dihydroxy-2,3-dihydrothiophene.

In the method of the production according to the present invention, a heterocyclic compound (substrate) and a hydroxylated heterocyclic compound (reaction product) can be preferably selected from the following combinations:

| Heterocyclic compound | Hydroxylated heterocyclic compound |
|---|---|
| 2-Phenyl quinoline | 3-(2-Quinolyl)-3,5-cyclohexadiene-1,2- |
| | diol |
| 2-Phenyl indole | 3-(1H-2-Indolyl)-3,5-cyclohexadiene- |
| | 1,2-diol |
| 2-Phenyl indole | 2-(1H-2-Indolyl)phenol |
| 2-Phenyl indole | 2-Phenyl-1H-5-indolol |
| 3-Phenyl-1-indanone | 3-(5,6-Dihydroxy-1,3-cyclohexadienyl)- |
| | 1-indanone |
| 2-Phenyl benzothiazole | 3-(1,3-Benzothiazole-2-yl)-3,5- |
| | cyclohexadiene-1,2-diol |
| 2-Phenyl benzoxazole | 3-(1,3-Benzoxazole-2-yl)-3,5- |
| | cyclohexadiene-1,2-diol |
| 2-Phenyl pyridine | 3-(2-Pyridyl)-3,5-cyclohexadiene-1,2- |
| | diol |
| 3-Metyl-2-phenyl | 3-(3-Methylpyrido-2-yl)-3,5- |
| pyridine | cyclohexadiene-1,2-diol |
| 4-Phenyl pyrimidine | 3-(4-Pyrimidinyl)-3,5-cyclohexadiene- |
| | 1,2-diol |
| 1-Phenyl pyrrole | 3-(1H-1-Pyrrolyl)-3,5-cyclohexadiene- |
| | 1,2-diol |
| 1-Phenyl pyrazole | 4-Hydroxy-1-phenylpyrazole |
| 3-Metyl-1-phenyl | 3-(3-Methylpyrazole-1-yl)-3,5- |
| pyrazole | cyclohexadiene-1,2-diol |
| 3-Metyl-1-phenyl | 2-(3-Methylpyrazole-1-yl)-phenol |
| pyrazole | |
| 2-Benzyl pyridine | 3-(2-Pyridylmethyl)-3,5- |
| | cyclohexadiene-1,2-diol |
| 1-Benzyl imidazole | 3-(1H-1-Imidazolylmethyl)-3,5- |
| | cyclohexadiene-1,2-diol |
| 4-Benzyl isothiazole | 3-(4-Isothiazolylmethyl)-3,5- |
| | cyclohexadiene-1,2-diol |
| 4-Benzyl isothiazole | 2-(4-Isothiazolylmethyl)phenol |
| 2-(2-Hydroxyphenyl)- | 2-(2-Hydroxyphenyl)-4,5-dihydro-1,3- |
| benzoxazole | benzoxazole-4,5-diol |
| 2-(p-Tolyl)pyridine | 2-(4-Methylphenyl)-3-pyridiol |
| 2-n-Butylbenzofuran | 2-Butylbenzo[b]furan-6-ol |
| 2-n-Butylbenzofuran | 2-Butylbenzo[b]furan-5-ol |
| 3-n-Hexyl thiophene | 4-Hexyl-2,3-dihydro-2,3-thiophenediol |

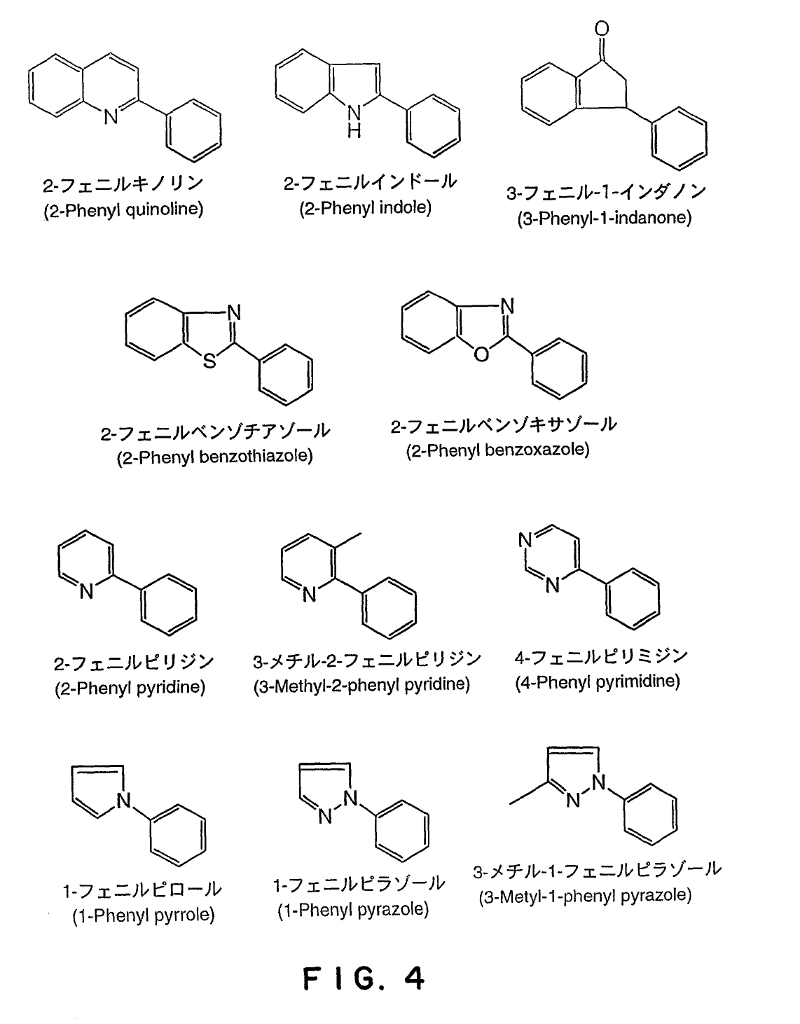

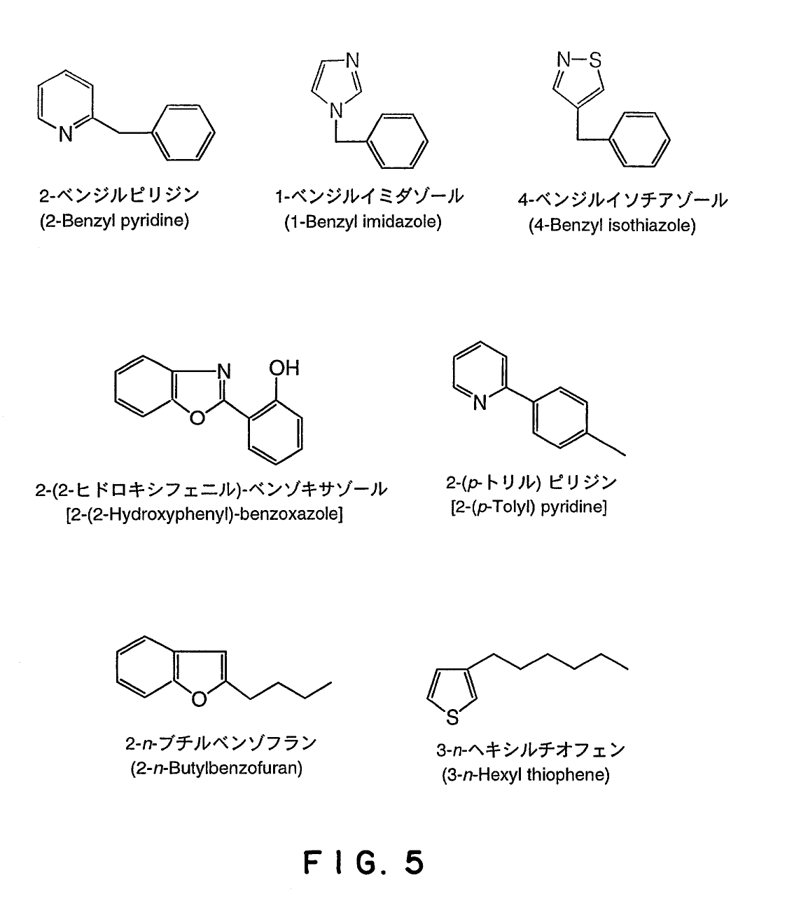

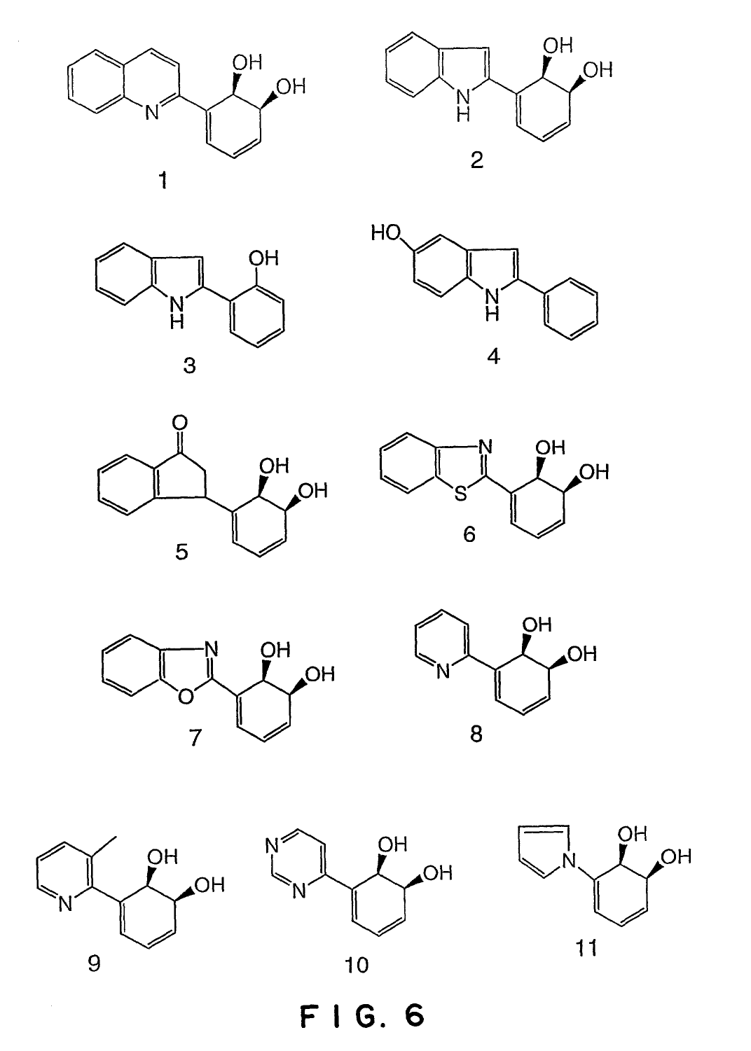

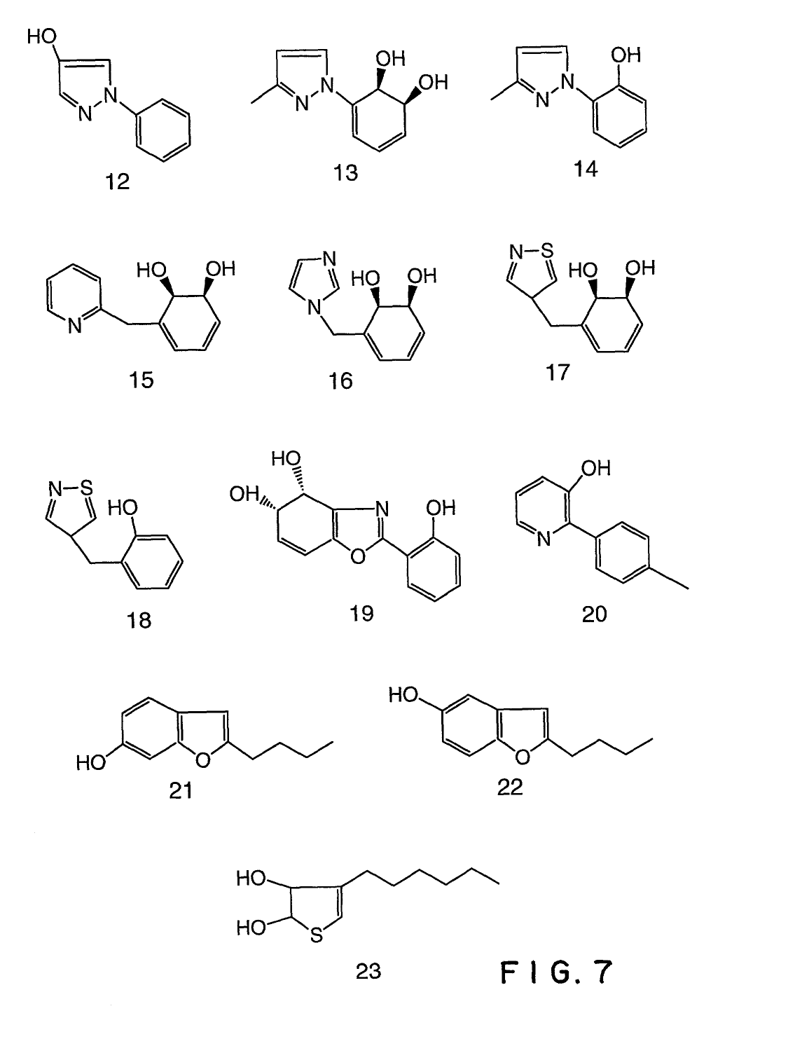

Chemical structures of the abovementioned heterocyclic compounds are shown in FIGS. 4 and 5. Absolute configurations of the abovementioned hydroxylated heterocyclic compounds are shown in FIGS. 6 and 7.

Further examples of heterocyclic compounds to be used in the present invention include flavonoids, such as flavone, flavanone, and 6-hydroxyflavanone. Flavonoids and hydroxylated flavonoids can be represented by the formula (I) and the formula (I′), respectively. In this case, Het in the formula (I) and the formula (I′) can be chromone (4H-chromene-4-on) or chroman-4-on, 6-hydroxy-chroman-4-on.

Examples of hydroxylated flavonoid include 2′,3′-dihydroxy derivatives, 2′-hydroxy derivatives, and 3′-hydroxy derivatives of flavonoid, such as 2′,3′-dihydroxyflavone, 3′-hydroxyflavone, 2′,3′-dihydroxyflavanone, 2′-hydroxyflavanone, 3′-hydroxyflavanone, 2′,6-dihydroxyflavanone, and 3′,6-dihydroxyflavanone.

In the method of the production according to the present invention, a flavonoid (substrate) and a hydroxylated flavonoid (reaction product) can be preferably selected from the following combinations.

| Flavonoid | Hydroxylated flavonoid |
|---|---|
| Flavone | 2′,3′-Dihydroxyflavone |
| Flavone | 3′-Hydoxyflavone |
| Flavanone | 2′,3′-Dihydroxyflavanone |
| Flavanone | 2′-Hydoxyflavanone |
| Flavanone | 3′-Hydoxyflavanone |
| 6-Hydroxyflavanone | 2′,6-Dihydroxyflavanone |
| 6-Hydroxyflavanone | 3′,6-Dihydroxyflavanone |

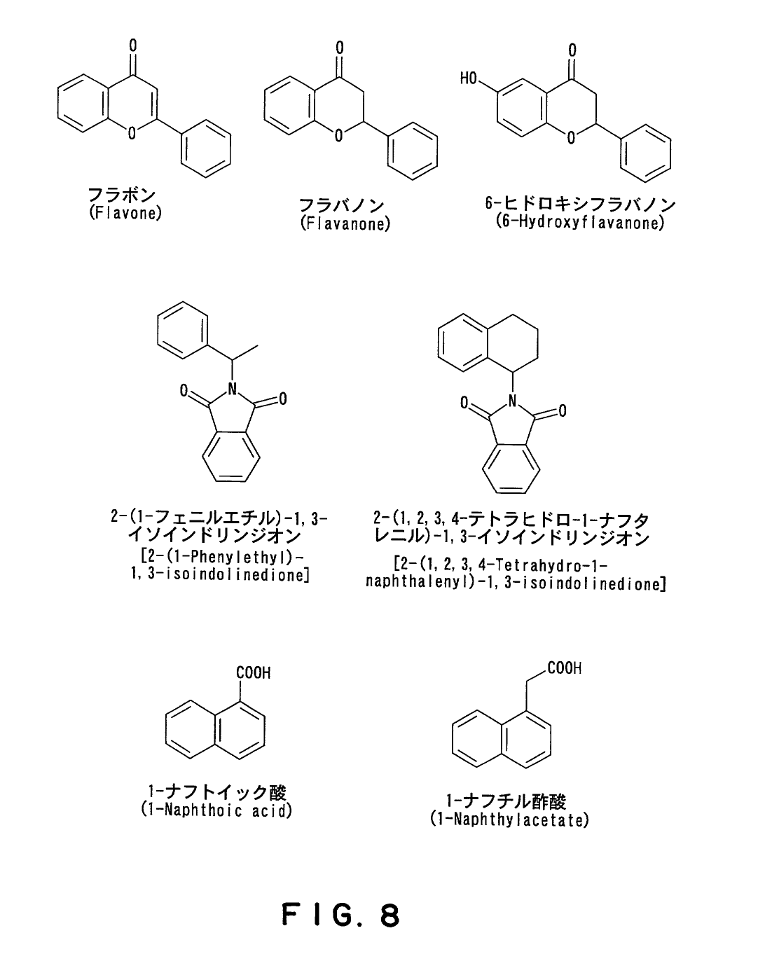

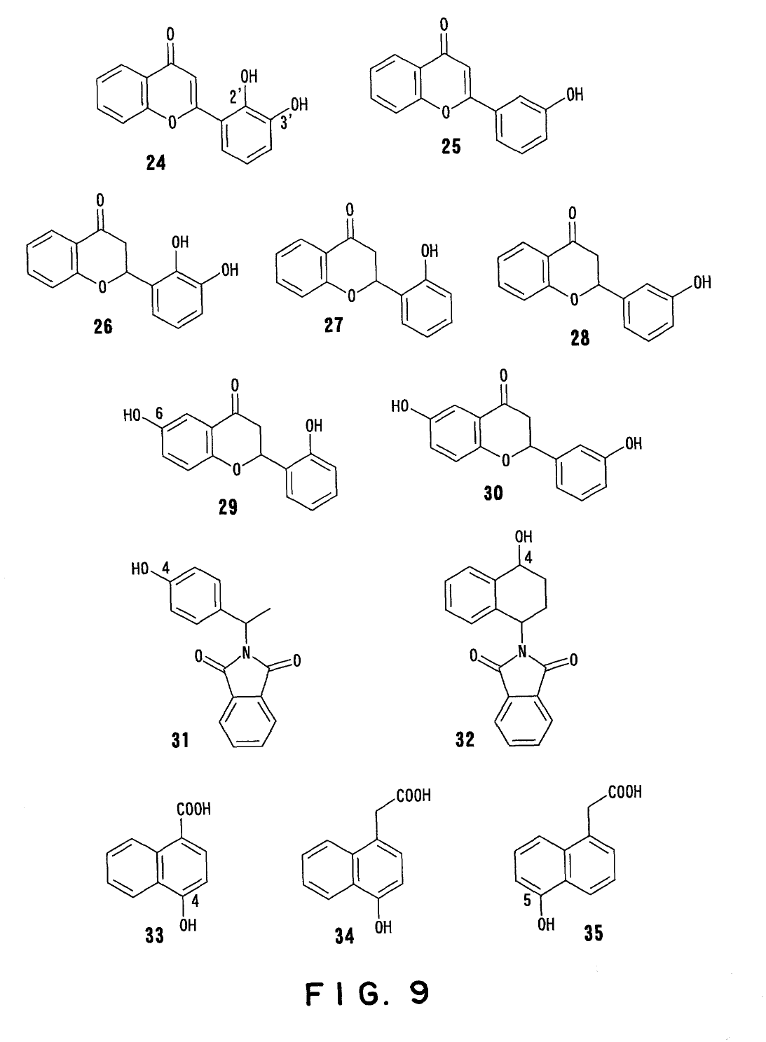

Chemical structures of the abovementioned flavonoids are shown in FIG. 8. Chemical structures of the abovementioned hydroxylated flavonoids are shown in FIG. 9.

Further examples of heterocyclic compounds to be used in the present invention include phthalimide derivatives having an aromatic ring, such as 2-(1-phenylethyl)-1, 3-isoindolinedione and 2-(1,2,3,4-tetrahydro-1-naphthalenyl)-1,3-isoindolinedione. Phthalimide derivatives having an aromatic ring and hydroxylated phthalimide derivatives having an aromatic ring are represented by the formula (I) and the formula (I′). In this case, Het in the formula (I) and the formula (I′) can be phthalimide.

Examples of hydroxylated phthalimide derivatives having an aromatic ring include hydroxylated phtalimide derivatives of which the aromatic ring or the benzyl group is hydroxylated, such as 2-[1-(4-hydroxyphenyl)ethyl]-1,3-isoindolinedione and 2-(4-hydroxy-1,2,3,4-tetrahydro-1-naphthalenyl)-1,3-isoindolinedione.

In the method of the production according to the present invention, a phthalimide derivative having an aromatic ring (substrate) and a hydroxylated phthalimide derivative having an aromatic ring (reaction product) are preferably selected from the following combinations.

| | Hydroxylated phthalimide |
|---|---|
| Phthalimide derivative | derivative having an aromatic |
| having an aromatic ring | ring |
| 2-(1-Phenylethyl)-1,3- | 2-[1-(4-Hydroxyphenyl)- |
| isoindolinedione | ethyl]-1,3-isoindolindione |
| 2-(1,2,3,4-Tetrahydro- | 2-(4-Hydroxy-1,2,3,4-tetra- |
| 1-naphthalenyl)-1,3- | hydro-1-naphthalenyl)-1,3- |
| isoindolinedione | isoindolinedione |

Chemical structures of the abovementioned aromatic phthalimide derivatives are shown in FIG. 8. Chemical structures of the abovementioned hydroxylated aromatic phthalimide derivatives are shown in FIG. 9.

Aromatic Carboxylic Acids and Hydroxylated Aromatic Carboxylic Acids

In the present specification, the term “aromatic carboxylic acid” refers to an aromatic compound having a carboxyl group in the molecule.

In the present specification, examples of the “aromatic compound” include benzene and naphthalene.

More specifically, an aromatic carboxylic acid can be represented by the formula (IV):

R³-Alkyl-COOR⁴ (IV)

wherein R³ is an unsubstituted carbon ring group, Alkyl is a bond or an optionally-branched alkylene chain having 1 to 4 carbon atoms, and R⁴is a hydrogen atom or a protecting group for a carboxyl group).

R³ is preferably an unsaturated 5- to 7-membered monocyclic carbon ring group or an unsaturated 9- to 11-membered bicyclic carbon ring group, more preferably phenyl or naphthyl.

A hydroxylated aromatic carboxylic acid can be represented by the formula (IV′):

R^{3′}-Alkyl-COOR⁴ (IV′)

(wherein Alkyl and R⁴ are the same as defined above, and R^{3′} is a carbon ring group substituted with 1 or 2 hydroxyl groups)

R^{3′} is preferably an unsaturated 5- to 7-membered monocyclic carbon ring group or an unsaturated 9- to 11-membered bicyclic carbon ring group, which is substituted with 1 or 2 hydroxyl groups, more preferably phenyl or naphthyl substituted with 1 or 2 hydroxyl groups.

Alkyl in the formula (IV) and the formula (IV′) is preferably a bond, methylene, or —(CH)(—CH₃)—.

In the method of the production according to the present invention, an aromatic carboxylic acid (substrate) and a hydroxylated aromatic carboxylic acid (reaction product) are selected from the following combinations:

| Aromatic carboxylic acid | Hydroxylated aromatic carboxylic acid |
|---|---|
| 1-Naphthoic acid | 4-Hydroxy-1-naphthoic acid |
| 1-Naphthylacetic acid | 4-Hydroxy-1-naphthylacetic acid |
| 1-Naphthylacetic acid | 5-Hydroxy-1-naphthylacetic acid |

Chemical structures of the abovementioned aromatic carboxylic acids are shown in FIG. 8. Chemical structures of the abovementioned hydroxylated aromatic carboxylic acids are shown in FIG. 9.

The biphenyl dioxygenase derived from Pseudomonas pseudoalcaligenes, in which the α-subunit is modified according to the aromatic ring dioxygenase derived from the strain Burkholderia cepacia LB400 (modified BphA1-BphA2BphA3-BphA4) can hydroxylate all of the abovementioned heterocyclic compounds.

A culture medium obtained by expressing the aromatic ring dioxygenase derived from the strain Pseudomonas pseudoalcaligenes KF707 (BphA1A2A3A4) can also convert various heterocyclic compounds. When the aromatic ring dioxygenase derived from the strain Pseudomonas pseudoalcaligenes KF707 is used, heterocyclic compounds other than 2-phenylpyridine, 3-methyl-2-phenylpyridine, 4-phenylpyrimidine, and 1-phenylpyrazole among the abovementioned heterocyclic compounds are preferable as a substrate.

The present invention provides a method for introducing a hydroxyl group into a heterocyclic compound. This method comprises the reaction of a heterocyclic compound with an aromatic ring dioxygenase. As described above, the aromatic ring dioxygenase includes (1) an aromatic ring dioxygenase derived from Pseudomonas pseudoalcaligenes and its modified form still having aromatic ring dioxygenase activity, and (2) an aromatic ring dioxygenase derived from Pseudomonas pseudoalcaligenes, of which α-subunit is modified according to the biphenyl dioxygenase derived from the strain Burkholderia cepacia LB400 (modified BphA1-BphA2-BphA3BphA4).

Furthermore, the present invention provides a composition to hydroxylate an heterocyclic compound. This composition comprises an aromatic ring dioxygenase. As mentioned above, the aromatic ring dioxygenase includes (1) an aromatic ring dioxygenase derived from Pseudomonas pseudoalcaligenes and its modified form still having aromatic ring dioxygenase activity, and (2) an aromatic ring dioxygenase derived from Pseudomonas pseudoalcaligenes, of which α-subunit is modified according to the biphenyl dioxygenase derived from the strain Burkholderia cepacia LB400 (modified BphA1-BphA2-BphA3-BphA4). The composition according to the present invention includes a composition comprising a liquid medium obtained by culturing microorganisms which express an aromatic ring dioxygenase, as well as a composition comprising an isolated and purified aromatic ring dioxygenase.

### EXAMPLE

The following Examples are presented to explain the present invention more specifically and should not be construed as limiting the scope of the invention.

Ordinary gene recombination experiments were performed in accordance with the standard method (Sambrook, J., Fritsch, E. F., Maniatis, T., “Molecular Cloning—A Laboratory Manual,” Cold Spring Harbor Laboratory Press, 1989), unless otherwise stated.

### Example 1

Construction of Expression Plasmid for E. coli1-1

Plasmid carrying the biphenyl dioxygenase gene derived from the strain Pseudomonas pseudoalcaligenes KF707.

The biphenyl dioxygenase gene group derived from Pseudomonas pseudoalcaligenes KF707 (bphA1A2A3A4) was inserted into E. coli vector pUC118 in the direction so that the inserted gene underwent the transcriptional read-through of the lac promoter to construct a plasmid, pKF6622, for biphenyl dioxygenase gene expression in E. coli. More specifically, a 6.78 kb XhoI fragment containing the bphA1A2A3A4-bphB-bphC gene group (see A. Suyama, R. Iwakiri, N. Kimura, A. Nishi, K. Nakamura, K. Furukawa, J. Bacteriol., 178, 4039-4046, 1996; or GenBank accession M83673) was inserted into the XhoI site of pUC118. Next, a 1.43 kb PpuMI fragment stretching over bphB and bphC was digested with PpuMI and then deleted by re-ligation. As a result, the plasmid pKF6622, in which a 5.35 kb fragment exclusively carrying the bphA1A2A3A4 gene was inserted in the direction so that the inserted gene underwent the transcriptional read-through of the lac promoter of pUC118, was obtained. A transformant (E. coli (pKF6622): FERM BP-7300) was obtained by inserting this pKF6622 into the strain E. coli JM109 and used for the following experiments.

1-2. Plasmid Containing Modified Biphenyl Dioxygenase Gene

A DNA (bphA1) encoding the large subunit of the biphenyl dioxygenase derived from the strain Burkholderia cepacia LB400 (the DNA sequence is registered at GenBank under the accession number M86348) and a DNA (bphA1) encoding the large subunit of the biphenyl dioxygenase derived from the strain Pseudomonas pseudoalcaligenes KF707 (the DNA sequence is registered at GenBank under the accession number M83673) were isolated by PCR using a bphA1 primer comprising a common flanking sequence. The bphA1 primer has a base sequence shown below.

A SacI site is located in the forward side, a BglII site is located in the reverse side (shown with italics), and furthermore, EcoRI sites are located-in both sides (shown with underlines). The PCR was conducted 25 cycles of 1 min at 94° C., 1.5 min at 52° C., and 1 min at 72° C.

| | |
|---|---|
| Forward: | 5′-CCGAATTCAAGGAGACGTTGAATCATGAGCTCAGC-3′ |
| Reverse: | 5′-TTGAATTCTTCCGGTTGACAGATCT-3′ |

The abovementioned two kinds of isolated bphA1s were mixed together and digested by treating with 0.15 unit of DNase I (Takara Shuzo) at 15° C. for 6 min. DNA fragments (10 to 50 bp) were recovered from agarose gel, mixed and subjected to self-priming PCR, PCR with the addition of the bphA1 primer to obtain PCR products containing various chimeric bphA1s in which the amino acid sequences were randomly exchanged (DNA shuffling). The PCR was carried out under the same conditions as described above, and the PCR products containing various chimeric bphA1s were double-digested with SacI/BglII, and then purified from the agarose gel.

In E. coil carrying the expression plasmid pJHF18 (see Hirose, J., Suyama, A., Hayashida, S., Furukawa, K., Gene, 128, 27-33, 1994) containing the bphA1A2A3A4-bphB-bphC gene group derived from the strain Pseudomonas-pseudoalcaligenes KF707, the reaction proceeds up to the meta-cleavage, which yields 2-hydroxy-6-oxo-6-phenylhexa-2,4-dienoic acid as a meta-cleavage product when biphenyl is used as a substrate. In general, meta-cleavage products can be monitored at 434 nm since they yield a yellow color. Since the plasmid pJHF18 has a single MluI site within the bphA1, a plasmid, pJHF18ΔMluI, in which only the bphA1 was disrupted, was constructed by carrying out digestion with MluI, filling-in and re-ligation (see T. Kumamura, H. Suenaga, M. Mitsuoka, T. Watanabe, K. Furukawa, Nature Biotechnology, 16, 663-666, 1998). Next, the pJHF18 MluI was double-digested with SacI/BglII to remove a 1.39 kb fragment containing the bphA1 gene only, and alternatively the PCR products containing the various chimeric bphA1s constructed above (double-digested with SacI/BalII) were inserted to obtain various plasmids (pSHF1000 series) containing various modified biphenyl dioxygenase genes (modified bphA1::bphA2A3A4 genes) and the bphBbphC gene. Biphenyl vapor was. -applied to E. coli XL1-Blue carrying these various plasmids and colonies showing a yellow color resulting from the meta-cleavage were selected and used for the following experiment. Colonies showing a yellow color resulting from the meta-cleavage indicate that the modified bphA1 genes obtained by the DNA shuffling can properly function.

One E. coli transformant (the plasmid contained in this E. coli was called pSHF1072) among several transformants, which were able to yield a yellow color with the biphenyl vapor, not only had a 2 times higher meta-cleavage decomposing efficiency than transformants having the bphA1 genes of corresponding parent strains (KF707 and LB400), but also had an ability to decompose benzene and toluene, which cannot be decomposed with the transformants having the bphA1 genes of corresponding parent strains. However, this decomposition efficiency was about ⅓ of the efficiency for those having a corresponding gene of P. putida F1, the todc1 gene,.

Next, the shuffled bphA1::bphA2A3A4 gene group contained in the plasmid pSHF1072 was inserted into the E. coli vector pUC118 in the direction so that the transcriptional read-through of the lac promoter underwent to construct plasmid pKF2072 for the expression of the modified biphenyl dioxygenase gene. More specifically, a 6.78 kb XhoI fragment containing the shuffled bphA1-bphA2A3A4-bphB-bphC gene group was excised from plasmid pSHF1072 and inserted into the XhoI site of pUC118. Next, a 1.43 kb PpuMI fragment stretching over bphB and bphc was deleted by PpuMI-digestion and re-ligation. As a result, a plasmid, pKF2072, was obtained, in which a 5.35 kb fragment exclusively carrying the shuffled bphA1 (derived from pSHF1072)::bphA2A3A4 gene, was inserted in the direction so that the transcriptional read-through of the lac promoter of pUC118 underwent. A transformant (E. coli (pKF2072): FERM BP-7299) was obtained by inserting this pKF2072 into the strain E. coli JM109 and used for the following experiments.

### Example 2

Co-cultivation of E. coli Transformant with Substrate

Cells of the recombinant E. coli carrying the two kinds of ferredoxin-associated aromatic ring dioxygenase genes constructed in Example 1, namely E. coli (pKF6622) and E. coli (pKF2072), were each cultured in LB liquid medium (1% tryptone, 0.5% yeast extract, 1% NaCl) containing 150 μg/ml ampicillin (Ap) up to the first half of exponential growth phase, the resulting culture was suspended in glycerol at a final concentration of about 30%, and then the suspension was placed in a deep freezer at −70° C. to −80° C. to obtain a glycerol stock culture. Further, as a control, cells of E. coli (JM 109 strain) having only the Ap resistant vector, such as pUC118, were cultured in the same manner to obtain a glycerol stock culture.

To start the conversion reaction, first, the necessary E. coli transformant cells were removed from the abovementioned glycerol stock culture with a platinum loop, suspended in 4 ml of LB medium containing 150 μg/ml ampicillin (Ap) and cultured at 28° C. for 7 to 8 hours at 175 rpm (pre-culture). Next, this pre-culture was placed in 70 ml of M9 medium containing 150 μg/ml Ap, 0.4% (w/v) glucose, and 10 μg/ml thiamine (see Sambrook, J., Fritsch, E. F., Maniatis, T., “Molecular cloning—A Laboratory Manual,” Cold Spring Harbor Laboratory Press, 1989, Appendix A-3), and the cultivation was carried out at 28° C. for 16 to 17 hours (overnight) at 175 rpm. The culture (main culture) reached an optical density (OD 600 nm) of about 1. The cells were recovered by centrifugation at 8,000 rpm for 5 minutes and suspended in 70 ml of M9 medium (supplemented with 150 μg/ml Ap, 0.4% (w/v) glucose, and 10 μg/ml thiamine) containing isopropyl-1-thio-β-D-galactopyranoside (IPTG) at a final concentration of 1 mM and 5 mg of a substrate, and the cultivation was further carried out at 28° C. for 2 to 3 days at 175 rpm. The substrate used was prepared by dissolving it in a solvent, such as ethanol, generally at a concentration of 10 mg/ml and added in an amount of 0.5 ml. On day 2 or 3 of the cultivation, the lipid was extracted by adding 70 ml of methanol and stirring for 30 minutes, and the supernatant was collected by centrifugation at 8,000 rpm for 5 minutes to obtain a crude lipid extract. The crude lipid extract was subjected to HPLC analysis immediately, although in most cases it could be stored at 4° C. for several weeks in this condition.

### Example 3

HPLC Analysis of Converted Product

The crude lipid extract prepared in Example 3 (80 μl) was used for a single injection. The HPLC analysis was carried out using a Puresil C18 column (4.6 mm×250 mm, Waters) at a rate of 1 ml/min. A Waters Alliance system was used as the main HPLC apparatus and a Waters 999 Model was used as a photodiode array detector. Conditions for development with solvents were as follows:

Solution A: water/methanol (50/50)

Solution B: methanol/2-propanol (60/40)

0 to 5 min (solution A), 5 to 20 min (solution A)→(solution B) convex gradient (No. 3, Waters), 20 min−(solution B)

Under these conditions, all compounds were generally isolated within 33 minutes. The conversion rate was expressed by a ratio of peak areas monitored at the wave length where the maximum absorbance was observed within the range from 230 to 350 nm (max plot).

When the conversion was confirmed by this analysis, the next purification and identification were performed. For this purification and identification process, cultivation was performed at 10 times the scale of Example 2.

### Example 4

Conversion Experiment using Various Substrates

Substrates used in the following experiments were purchased from Sigma-Aldrich, Tokyo Kasei and the like.

4-1. Conversion of 2-phenylquinoline

A conversion experiment for 2-phenylquinoline (FIG. 4) was carried out using two kinds of the recombinant E. coli (including control) according to the method described in Example 2. Co-cultivation was carried out by adding 0.5 ml of 2-phenylquinoline dissolved in ethanol at a concentration of 10 mg/ml to 70 ml of the main culture medium. Results of the HPLC analysis revealed that E. coli (pKF2072) and E. coli (pKF6622) were able to utilize and convert 2-phenylquinoline as a substrate. The conversion rates were 89% and 53%, respectively.

4-2. Conversion of 2-phenylindole

A conversion experiment for 2-phenyl indole (FIG. 4) was carried out using two kinds of the recombinant E. coli (including control) according to the method described in Example 2. Co-cultivation was carried out by adding 0.5 ml of 2-phenylindole dissolved in ethanol at a concentration of 10 mg/ml to 70 ml of the main culture medium. Results of the HPLC analysis revealed that E. coli (pKF2072) and E. coli (pKF6622) were able to utilize and convert 2-phenyl indole as a substrate. The conversion rates were 71% and 23%, respectively. Three conversion product peaks were observed for E. coli (pKF2072).

4-3. Conversion of 3-phenyl-1-indanone

A conversion experiment for 3-phenyl-1-indanone (FIG. 4) was carried out using two kinds of the recombinant E. coli (including control) according to the method described in Example 2. Co-cultivation was carried out by adding 0.5 ml of 3-phenyl-1-indanone dissolved in ethanol at a concentration of 10 mg/ml to 70 ml of the main culture medium. Results of the HPLC analysis revealed that E. coli (pKF2072) and E. coli (pKF6622) were able to utilize and convert 3-phenyl-1-indanone as a substrate. The conversion rates were 97% and 93%, respectively.

4-4. Conversion of 2-phenylbenzothiazole

A conversion experiment for 2-phenylbenzothiazole (FIG. 4) was carried out using two kinds of the recombinant E. coli (including control) according to the method described in Example 2. Co-cultivation was carried out by adding 0.5 ml of 2-phenylbenzothiazole dissolved in ethanol at a concentration of 10 mg/ml to 70 ml of the main culture medium. Results of the HPLC analysis revealed that E. coli (pKF2072) and E. coli (pKF6622) were able to utilize and convert 2-phenyl benzothiazole as a substrate. The conversion rates were 81% and 36%, respectively.

4-5. Conversion of 2-phenylbenzoxazole

A conversion experiment for 2-phenylbenzoxazole (FIG. 4) was carried out using two kinds of the recombinant E. coli (including control) according to the method described in Example 2. Co-cultivation was carried out by adding 0.5 ml of 2-phenylbenzoxazole dissolved in ethanol at a concentration of 10 mg/ml to 70 ml of the main culture medium. Results of the HPLC analysis revealed that E. coil (pKF2072) and E. coli (pKF6622) were able to utilize and convert 2-phenyl benzoxazole as a substrate. The conversion rates were 100% and 45%, respectively.

4-6. Conversion of 2-phenylpyridine

A conversion experiment for 2-phenylpyridine (FIG. 4) was carried out using two kinds of the recombinant E. coli (including control) according to the method described in Example 2. Co-cultivation was carried out by adding 0.5 ml of 2-phenylpyridine dissolved in 70% ethanol at a concentration of 10 mg/ml to 70 ml of the main culture medium. Results of the HPLC analysis revealed that only E. coli (pKF2072) was evidently able to utilize and convert 2-phenyl pyridine as a substrate. The conversion rate was 14%. Conversion products were hardly observed for E. coli (pKF6622).

4-7. Conversion of 3-methyl-2-phenylpyridine

A conversion experiment for 3-methyl-2-phenylpyridine (FIG. 4) was carried out using two kinds of the recombinant E. coli (including control) according to the method described in Example 2. Co-cultivation was carried out by adding 0.5 ml of 3-methyl-2-phenylpyridine dissolved in 70% ethanol at a concentration of 10 mg/ml to 70 ml of the main culture medium. Results of the HPLC analysis revealed that only E. coli (pKF2072) was evidently able to utilize and convert 3-methyl-2-phenyl pyridine as a substrate. The conversion rate was 16%. Conversion products were hardly observed for E. coli (pKF6622).

4-8. Conversion of 4-phenylpyrimidine

A conversion experiment for 4-phenylpyrimidine (FIG. 4) was carried out using two kinds of the recombinant E. coli (including control) according to the method described in Example 2. Co-cultivation was carried out by adding 0.5 ml of 4-phenylpyrimidine dissolved in 70% ethanol at a concentration of 10 mg/ml to 70 ml of the main culture medium. Results of the HPLC analysis revealed that only E. coli (pKF2072) was evidently able to utilize and convert 4-phenyl pyrimidine as a substrate. The conversion rate was 100%. Conversion products were hardly observed for E. coli (pKF6622).

4-9. Conversion of 1-phenylpyrrole

A conversion experiment for 1-phenylpyrrole (FIG. 4) was carried out using two kinds of the recombinant E. coli (including control) according to the method described in Example 2. Co-cultivation was carried out by adding 0.5 ml of 1-phenylpyrrole dissolved in ethanol at a concentration of 10 mg/ml to 70 ml of the main culture medium. Results of the HPLC analysis revealed that E. coli (pKF2072) and E. coli (pKF6622) were able to utilize and convert 1-phenylpyrrole as a substrate. The conversion rate was 100% for both recombinants.

4-10. Conversion of 1-phenylpyrazole

A conversion experiment for 1-phenyl pyrazole (FIG. 4) was carried out using two kinds of the recombinant E. coli (including control) according to the method described in Example 2. Co-cultivation was carried out by adding 0.5 ml of 1-phenylpyrazole dissolved in 70% ethanol at a concentration of 10 mg/ml to 70 ml of the main culture medium. Results of the HPLC analysis revealed that only E. coli (pKF2072) was evidently able to utilize and convert 1-phenyl pyrazole as a substrate. The conversion rate was 47%. Conversion products were hardly observed for E. coli (pKF6622).

4-11. Conversion of 3-methyl-1-phenylpyrazole

A conversion experiment for 3-methyl-1-phenylpyrazole (FIG. 4) was carried out using two kinds of the recombinant E. coli (including control) according to the method described in Example 2. Co-cultivation was carried out by adding 0.5 ml of 3-methyl-1-phenylpyrazole dissolved in ethanol at a concentration of 10 mg/ml to 70 ml of the main culture medium. Results of the HPLC analysis revealed that E. coli (pKF2072) and E. coli (pKF6622) were able to utilize and convert 3-methyl-1-phenylpyrazole as a substrate. The conversion rates were 100% and 63%, respectively. Two conversion product peaks were observed for E. coli (pKF2072).

4-12. Conversion of 2-benzylpyridine

A conversion experiment for 2-benzylpyridine (FIG. 5) was carried out using two kinds of the recombinant E. coli (including control) according to the method described in Example 2. Co-cultivation was carried out by adding 0.5 ml of 2-benzylpyridine dissolved in ethanol at a concentration of 10 mg/ml to 70 ml of the main culture medium. Results of the HPLC analysis revealed that E. coli (pKF2072) and E. coli (pKF6622) were able to utilize and convert 2-benzylpyridine as a substrate. The conversion rates were 57% and 11%, respectively.

4-13. Conversion of 1-benzylimidazole

A conversion experiment for 1-benzylimidazole (FIG. 5) was carried out using two kinds of the recombinant E. coli (including control) according to the method described in Example 2. Co-cultivation was carried out by adding 0.5 ml of 1-benzylimidazole dissolved in ethanol at a concentration of 10 mg/ml to 70 ml of the main culture medium. Results of the HPLC analysis revealed that E. coli (pKF2072) and E. coli (pKF6622) were able to utilize and convert 1-benzylimidazole as a substrate. The conversion rates were 97% and 43%, respectively.

4-14. Conversion of 4-benzylisothiazole

A conversion experiment for 4-benzylisothiazole (FIG. 5) was carried out using two kinds of the recombinant E. coli (including control) according to the method described in Example 2. Co-cultivation was.carried out by adding 0.5 ml of 4-benzylisothiazole dissolved in ethanol at a concentration of 10 mg/ml to 70 ml of the main culture medium. Results of the HPLC analysis revealed that E. coli (pKF2072) and E. coli (pKF6622) were able to utilize and convert 4-benzylisothiazole as a substrate. The conversion rates were 65% and 27%, respectively. Two conversion product peaks were observed for E. coli (pKF2072).

4-15. Conversion of 2-(2-hydroxyphenyl)-benzoxazole

A conversion experiment for 2-(2-hydroxyphenyl)-benzoxazole (FIG. 5) was carried out using two kinds of the recombinant E. coli (including control) according to the method described in Example 2. Co-cultivation was carried out by adding 1 ml of 2-(2-hydroxyphenyl)-benzoxazole dissolved in ethanol at a concentration of 5 mg/ml to 70 ml of the main culture medium. Results of the HPLC analysis revealed that E. coli (pKF2072) and E. coli (pKF6622) were able to utilize and convert 2-(2-hydroxyphenyl)-benzoxazole as a substrate. The conversion rates were 39% and 25%, respectively.

4-16. Conversion of 2-(p-tolyl)pyridine.

A conversion experiment for 2-(p-tolyl)pyridine (FIG. 5) was carried out using two kinds of the recombinant E. coil (including control) according to the method described in Example 2. Co-cultivation was carried out by adding 0.5 ml of 2-(p-tolyl)pyridine dissolved in 70% ethanol at a concentration of 10 mg/ml to 70 ml of the main culture medium. Results of the HPLC analysis revealed that E. coli (pKF2072) and E. coli (pKF6622) were able to utilize and convert 2-(p-tolyl)pyridine as a substrate. The conversion rates were 96% and 61%, respectively.

4-17. Conversion of 2-n-butylbenzofuran

A conversion experiment for 2-n-butylbenzofuran (FIG. 5) was carried out using two kinds of the recombinant E. coli (including control) according to the method described in Example 2. Co-cultivation was carried out by adding 0.5 ml of 2-n-butylbenzofuran dissolved in ethanol at a concentration of 10 mg/ml to 70 ml of the main culture medium. Results of the HPLC analysis revealed that E. coli (pKF2072) and E. coli (pKF6622) were able to utilize and convert 2-n-butylbenzofuran as a substrate. The conversion rates were 100% and 90%, respectively. Two conversion product peaks were observed for E. coli (pKF2072).

4-18. Conversion of 3-n-hexylthiophene

A conversion experiment for 3-n-hexylthiophene (FIG. 5) was carried out using two kinds of the recombinant E. coli (including control) according to the method described in Example 2. Co-cultivation was carried out by adding 0.5 ml of 3-n-hexylthiophene dissolved in ethanol at a concentration of 10 mg/ml to 70 ml of the main culture medium. Results of the HPLC analysis revealed that E. coli (pKF2072) and E. coli (pKF6622) were able to utilize and convert 3-n-hexylthiophene as a substrate. The conversion rates were 100% and 99%, respectively.

### Example 5

Purification and Identification of Converted Products

5-1. Conversion product of 2-phenylquinoline (FIG. 6)

To a mixed culture fluid of E. coli (pKF2072) and 2-phenylquinoline (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 55 mg of an extract which contained products. The extract was applied onto a silica gel column (Merck 60, Ø 2 cm×30 cm) and then developed with a solvent, hexane:ethyl acetate=10:1, to isolate compound 1 (3-(2-quinolyl)-3,5-cyclohexadiene-1,2-diol) (12 mg) as a pure substance.

Physical characteristics of compound 1 (3-(2-quinolyl)-3,5-cyclohexadiene-1,2-diol)

EI-MS(m/z): 239(M⁺)

¹H-NMR: (500 MHz, CDCl₃): 4.50(dd,J=3.0,6.7,1H), 5.08(d,J=6.7,1H), 6.23(m,2H), 6.78(m,1H), 7.46(dd,J=6.7,6.7,1H), 7.49(dd,J=6.7,6.7,1H), 7.69(d,J=6.7,1H), 7.73(d,J=6.7,1H), 7.96(d,J=8.5,1H), 8.07(d,J=9.2,1H)

5-2. Converted products of 2-phenylindole (FIG. 6)

To a mixed culture fluid of E. coli (pKF2072) and 2-phenylindole (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 86 mg of an extract which contained products. The extract was applied onto a silica gel column (Merck 60, Ø2 cm×30 cm) and then developed with a solvent, hexane:ethyl acetate=10:1, to isolate compound 2 (3-(1H-2-indolyl)-3,5-cyclohexadiene-1,2-diol) (5 mg), compound 3 (2-(1H-2-indolyl)phenol) (10 mg), and compound 4 (2-phenyl-1H-5-indolol) (7 mg) each as a pure substance.

Physical characteristics of compound 2 (3-(1H-2-indolyl)-3,5-cyclohexadiene-1,2-diol)

EI-MS(m/z):225(M⁺)

¹H-NMR(500 MHz, DMSO-d₆) 4.34(2H), 4.70(d,J=5.5,1H), 4.97(d,J=6.0,1H), 5.78(d,J=9.2,1H), 6.01(ddd,J=2.4,5.5,9.2), 6.50(d,J=5.5,1H), 6.61(s,1H), 6.94(dd,J=7.9,7.9,1H), 7.06(dd,J=7.9,7.9,1H), 7.30(d,J=7.3,1H), 7.47(d,J=7.3,1H), 11.11(s,1H)

Physical characteristics of compound 3 (2-(1H-2-indolyl)phenol)

EI-MS(m/z): 209(M⁺)

¹H-NMR: (500 MHz, CDCl₃): 5.63(brs,1H), 6.84(d,J=2.0,1H), 6.90(d,J=8.5,1H), 7.02(dd,J=7.3,7.3,1H), 7.12(dd,J=7.3,7.3,1H), 7.16-7.22(3H), 7.40(d,J=8.5,1H), 7.63(d,J=7.9,1H), 7.67(dd,J=2.0,7.9,1H)

Physical characteristics of compound 4 (2-phenyl1H5-indolol)

EI-MS(m/z):209(M⁺)

¹H-NMR:(500 MHz, CDCl₃): 6.60(dd,J=2.4,8.5,1H), 6.70(d,J=2.0,1H), 6.82(d,J=2.4,1H), 7.17(d,J=8.5,1H), 7.27(dd,J=7.3,7.3,1H), 7.42(dd,J=7.3,7.3,2H), 7.79(d,J=7.3,2H), 8.66(brs,1H), 11.19(s,1H)

5-3. Converted product of 3-phenyl-1-indanone (FIG. 6)

To a-mixed culture fluid of E. coli (pKF2072) and 3-phenyl-1-indanone (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 57 mg of an extract which contained products. The extract was applied onto a silica gel column (Merck 60, Ø 2 cm×30 cm) and then developed with a solvent, dichloromethane:ethyl acetate=2:1, to isolate compound 5 (3-(5,6-dihydroxy-1,3-cyclohexadienyl)-1-indanone)(10 mg) as a pure substance.

Physical characteristics of compound 5 (3-(5,6dihydroxy-1,3-cyclohexadienyl)-1-indanone)

EI-MS(m/z): 242(M⁺)

¹H-NMR: (500 MHz, CDCl₃): 2.68(dd,J=3.1,18.9,1H), 3.01(dd,J=7.9,18.9,1H), 4.17-4.28(3H), 5.70(d,J=4.9,1H), 5.91(m,1H), 5.95(m,1H), 7.38(dd,J=7.3,7.3,1H), 7.45(d,J=7.3,1H), 7.58(dd,J=7.3,7.3,1H), 7.74(d,J=7.3,1H)

5-4. Converted product of 2-phenylbenzothiazole (FIG. 6)

To a mixed culture fluid of E. coli (pKF2072) and 2-phenylbenzothiazole (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 68 mg of an extract which contained products. The extract was applied onto a silica gel column (Merck 60, Ø 2 cm×30 cm) and then developed with a solvent, dichloromethane:ethyl acetate=5:1, to isolate compound 6 (3-(1,3-benzothiazol-2-yl)-3,5-cyclohexadiene-1,2-diol) (32.5 mg) as a pure substance.

Physical characteristics of compound 6 (3-(1,3-benzothiazol-2-yl)-3,5-cyclohexadiene-1,2-diol)

EI-MS(m/z): 245(M⁺)

¹H-NMR: (500 MHz, CDCl₃): 4.51(m,1H), 5.00(d,J=6.1,1H), 6.21(dd,J=4.9,9.2,1H), 6.26(dd,J=4.3,9.2,1H), 6.78(d,J=4.9,1H), 7.34(dd,J=7.3,7.3,1H), 7.44(dd,J=7.3,7.3), 7.81(d,J=7.3,1H), 7.94(d,J=7.3,1H)

5-5. Converted product of 2-phenylbenzoxazole (FIG. 6)

To a mixed culture fluid of E. coli (pKF2072) and 2-phenylbenzoxazole (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 65.4 mg of an extract which contained products. The extract was applied onto a silica gel column (Merck 60, Ø 2 cm×30 cm) and then developed with a solvent, CH₂Cl₂:ethyl acetate=1:1, to isolate compound 7 (3-(1,3-benzoxazol-2-yl)-3,5-cyclohexadiene-1,2-diol) (26.1 mg) as a pure substance.

Physical characteristics of compound 7 (3-(1,3-benzoxazol-2-yl)-3,5-cyclohexadiene-1,2-diol)

EI-MS(m/z):229(M⁺)

¹H-NMR: (500 MHz, DMSO-d₆) 4.41(m,1H), 4.62(dd,J=5.5, 5.5,1H), 4.97(d,J=5.5,1H), 5.18(d,J=7.1,1H), 6.08-6.15(2H), 7.10(d,J=4.9,1H), 7.33-7.40(2H), 7.68(dd,J=2.0, 6.7,1H), 7.72(dd,J=2.0, 6.7,1H)

5-6. Converted product of 2-phenylpyridine (FIG. 6)

To a mixed culture fluid of E. coli (pKF2072) and 2-phenylpyridine (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 50 mg of an extract which contained products. The extract was applied onto a silica gel column (Merck 60, Ø 2 cm×30 cm) and then developed with a solvent, CH₂Cl₂:MeOH=40:1, to isolate compound 8 (3-(2-pyridyl)-3,5-cyclohexadiene-1,2-diol) (10 mg) as a pure substance.

Physical characteristics of compound 8 (3-(2-pyridyl)-3,5-cyclohexadiene-1,2-diol)

EI-MS(m/z) 189(M⁺)

¹H-NMR (500 MHz, DMSO-d₆) 4.34(dd,J=2.5,5.5,1H), 4.56(d,J=5.5,1H), 5.89(d,J=10.2,1H), 6.04(ddd,J=3.0,5.5,9.8), 6.92(d,J=5.5,1H), 7.21(dd,J=4.9,8.0,1H), 7.63(d,J=8.0,1H), 7.75(dd,J=8.0,8.0,1H), 8.54(d,J=4.9,1H)

5-7. Converted product of 3-methyl-2-phenylpyridine (FIG. 6)

To a mixed culture fluid of E. coli (pKF2072) and 3-methyl-2-phenylpyridine (70 ml), an equal volume of methanol was added and the admixture was stirred at room temperature for 2 hours. An HPLC analysis was performed using 70 μl of this admixture. A product peak was found at a retention time of 5.88 min and showed the maximum absorption (λₘₐₓ) at 290 nm. The structure of compound 9 was determined to be 3-(3-methylpyrid-2-yl)-3,5-cyclohexadiene-1,2-diol by a comparison with the absorption spectrum (λₘₐₓ=295 nm) for compound 8 and the retention time.

5-8. Conversion product of 4-phenylpyridine (FIG. 6)

To a mixed culture fluid of E. coli (pKF2072) and 4-phenylpyridine (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 23.5 mg of an extract which contained products. The extract was applied onto a silica gel column (Merck 60, Ø 2 cm×30 cm) and then developed with a solvent, CH₂Cl₂:MeOH=30:1, to isolate compound 10 (3-(4-pyrimidinyl)-3,5-cyclohexadiene-1,2-diol) (6.6 mg) as a pure substance.

Physical characteristics of compound 10 (3-(4-pyrimidinyl)-3,5-cyclohexadiene-1,2-diol)

EI-MS:190(M⁺)

¹H-NMR:(500 MHz, CDCl₃) 4.54(d,J=6.0,1H), 4.84(d,J=6.0,1H), 6.16-6.24(2H), 6.91(d,J=4.9,1H), 7.52(dd,J=1.8,5.5,1H), 8.66(d,J=5.5,1H), 9.11(d,J=1.8,1H)

5-9. Converted product of 1-phenylpyrrole (FIG. 6)

To a mixed culture fluid of E. coli (pKF2072) and 1-phenylpyrrole (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 25 mg of an extract which contained products. The extract was applied onto a silica gel column (Merck 60, Ø 2 cm×30 cm) and then developed with a solvent, hexane:ethyl acetate=10:1, to isolate compound 11 (3-(1H-1-pyrrolyl)-3,5-cyclohexadiene-1,2-diol) (5 mg) as a pure substance.

Physical characteristics of compound 11 (3-(1H-1-pyrrolyl)-3,5-cyclohexadiene-1,2-diol)

EI-MS(m/z): 163(M⁺)

¹H-NMR: (500 MHz, CDCl₃): 4.44(d,J=6.1,1H), 4.62(ddd,J=3.0,3.0,6.1,1H), 5.71(dd,J=2.4,9.8,1H), 5.91(d,J=6.1,1H), 5.97(ddd,J=2.4,6.1,9.8,1H), 6.26(dd,J=2.4,2.4,2H), 6.99(dd J=2.4,2.4,2H)

5-10. Converted product of 1-phenylpyrazole (FIG. 7)

To a mixed culture fluid of E. coli (pKF2072) and 1-phenylpyrazole (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 55 mg of an extract which contained products. The extract was applied onto a silica gel column (Merck 60, Ø 2 cm×30 cm) and then developed with a solvent, hexane:ethyl acetate=2:1, to isolate compound 12 (4-hydroxy-1-phenyl pyrazole) (7.0 mg) as a pure substance.

Physical characteristics of compound 12 (4-hydroxy-1-phenyl pyrazole)

EI-MS(m/z): 160

¹H-NMR: (500 MHz, CDCl₃) 7.16-7.22(1H), 7.34-7.38(3H), 7.50-7.55(3H)

5-11. Converted products of 3-methyl-1-phenylpyrazole (FIG. 7)

To a mixed culture fluid of E. coli (pKF2072) and 3-methyl-1-phenylpyrazole (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 68 mg of an extract which contained products. The extract was applied onto a silica gel column (Merck 60, Ø 2 cm×30 cm) and then developed with a solvent, CH₂Cl₂:MeOH=20:1 (stepwise), to isolate compound 13 (3-(3-methylpyrazol-1-yl)-3,5-cyclohexadiene-1,2-diol) (18 mg) and compound 14 (2-(3-methylpyrazol-1-yl)-phenol) (19 mg) as a pure substance.

Physical characteristics of compound 13 (3-(3-methylpyrazol-1-yl)-3,5-cyclohexadiene-1,2-diol)

EI-MS(m/z):192(M⁺)

¹H-NMR:(500 MHz, CDCl₃) 2.28(s,3H), 4.54(m,1H), 4.81(d,J=6.0,1H), 5.86(dd,J=3.0,5.0,1H), 6.00-6.08(2H), 6.15(d,J=2.5,1H), 7.65(d,J=2.5,1H)

Physical characteristics of compound 14 (2-(3-methylpyrazol-1-yl)-phenol)

EI-MS(m/z):174(M⁺)

¹H-NMR:(500 MHz, CDCl₃) 2.31(s,3H), 6.20(d,J=2.5,1H), 6.82(dd,J=7.4,7.4,1H), 7.03(d,J=7.4,1H), 7.09(dd,J=7.4,7.4,1H), 7.25(d,J=7.4,1H), 7.81(d,J=2.5,1H), 11.53(s,1H)

5-12. Converted product of 2-benzylpyridine (FIG. 7)

To a mixed culture fluid of E. coli (pKF2072) and 2-benzylpyridine (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 55 mg of an extract which contained products. The extract was applied onto a silica gel column (Merck 60, Ø 2 cm×30 cm) and then developed with a solvent, CH₂Cl₂:MeOH=50:1, to isolate compound 15 (3-(2-pyridylmethyl)-3,5-cyclohexadiene-1,2-diol (5.6 mg) as a pure substance.

Physical characteristics of compound 15 (3-(2-pyridylmethyl)-3,5-cyclohexadiene-1,2-diol)

EI-MS(m/z): 203(M⁺)

¹H-NMR (500 MHz,DMSO-d₆) 3.55-3.62(2H), 3.82(m,1H), 4.03(m,1H), 5.57(d,J=4.9,1H), 5.66(dd,J=3.0, 9.7,1H), 5.80(m,1H), 7.21(dd,J=5.5, 6.0,1H), 7.27(d,J=7.6,1H), 7.70(ddd,J=4.9, 6.0, 7.6,1H), 8.46(d,J=5.5,1H)

5-13. Converted product of 1-benzylimidazole (FIG. 7)

To a mixed culture fluid of E. coli (pKF2072) and 1-benzylimidazole (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 65 mg of an extract which contained products. The extract was applied onto a silica gel column (Merck 60, Ø 2 cm×30 cm) and then developed with a solvent, CH₂Cl₂:MeOH=7:1, to isolate compound 16 (3-(1H-imidazolylmethyl)-3,5-cyclohexadiene-1,2-diol) (2 mg) as a pure substance.

Physical characteristics of compound 16 (3-(1H-imidazolylmethyl)-3,5-cyclohexadiene-1,2-diol)

EI-MS(m/z):192(M⁺)

¹H-NMR(500 MHz,DMSO-d₆) 3.72(m,1H), 4.00(m,1H), 4.62(d,J=15.9,1H), 4.77(d,J=15.9,1H), 5.58(d,J=5.5,1H), 5.75(dd,J=3.0, 9.1,1H), 5.82(m,1H), 6.89(s,1H), 7.10(s,1H), 7.61(s,1H)

5-14. Converted products of 4-benzylisothiazole (FIG. 7)

To a mixed culture fluid of E. coli (pKF2072) and 1-benzylisothiazole (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 34 mg of an extract which contained products. The extract was applied onto a silica gel column (Merck 60, Ø 2 cm×30 cm) and then developed with a solvent, CH₂Cl₂:MeOH=40:1, to isolate compound 17 (3-(4-isothiazolylmethyl)-3,5-cyclohexadiene-1,2(8.0 mg) and compound 18 (2-(4-isothiazolylmethyl)-phenol) (6.5 mg) as a pure substance.

Physical characteristics of compound 17 (3-(4-isothiazolylmethyl)-3,5-cyclohexadiene-1,2-diol)

EI-MS(m/z):209(M⁺)

¹H-NMR:(500 MHz, DMSO-d₆) 3.52(d,J=16.5,1H), 3.62(d,J=16.5,1H), 3.78(dd,J=6.0,6.0,1H), 4.03(m,1H), 4.61(d,J=6.7,1H), 4.66(d,J=6.0,1H), 5.55(d,J=5.5,1H), 5.68(dd,J=3.0,9.8,1H), 5.80(dd,J=5.5,9.8,1H), 8.42(s,1H), 8.70(s,1H)

Physical characteristics of compound 18 (2-(4-isothiazolylmethyl)-phenol)

EI-MS(m/z):191(M⁺)

¹H-NMR:(500 MHz, DMSO-d₆) 3.93(s,2H), 6.71(dd,J=7.4,7.4,1H), 6.80(d,J=7.4,1H), 7.02(dd,J=7.4,1H), 7.06(d,J=7.4,1H), 8.43(s,1H), 8.59(s,1H), 8.72(s,1H)

5-15. Converted product of 2-(2-hydroxyphenyl)benzoxazole (FIG. 7)

To a mixed culture fluid of E. coli (pKF2072) and 2-(2-hydroxyphenyl)benzoxazole (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 40 mg of an extract which contained products. The extract was applied onto a silica gel column (Merck 60, Ø 2 cm×30 cm) and then developed with a solvent, dichloromethane:ethyl acetate=10:1, to isolate compound 19 (2-(2-hydroxyphenyl)-4,5-dihydro-1,3-benzoxazole-4,5-diol) (7.8 mg) as a pure substance.

Physical characteristics of compound 19 (2-(2-hydroxyphenyl)-4,5-dihydro-1,3-benzoxazole-4,5-diol)

EI-MS(m/z): 243(M⁺)

¹H-NMR: (500 MHz, DMSO-d₆): 4.50(2H), 5.22(d,J=5.5,1H), 5.33(d,J=6.7,1H), 5.95(d,J=10.0,1H), 6.57(dd,J=2.4,10.0,1H), 7.00(dd,J=7.3,7.3,1H), 7.04(d,J=8.6,1H), 7.39(dd,J=7.3,8.6,1H), 7.79(d,J=7.3,1H), 10.92(s,1H)

5-16. Converted product of 2-(p-tolyl)pyridine (FIG. 7)

To a mixed culture fluid of E. coli (pKF2072) and 2-(p-tolyl)pyridine (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 65 mg of an extract which contained products. The extract was applied onto a silica gel column (Merck 60, Ø 2 cm×30 cm) and then developed with dichloromethane to isolate compound 20 (2-(4-methylphenyl)-3-pyridiol) (9 mg) as a pure substance.

Physical characteristics of compound 20 (2-(4-methylphenyl)-3-pyridiol)

EI-MS(m/z): 185(M⁺)

¹H-NMR: (500 MHz, DMSO-d₆): 2.33(s,3H), 7.15(dd,J=4.3,7.9,1H), 7.21(d,J=7.9,2H), 7.29(d,J=7.9,1H), 7.91(d,J=7.9,2H), 8.11(d,J=4.3,1H), 10.06(s,1H)

5-17. Converted products of 2-n-butylbenzofuran (FIG. 7)

To a mixed culture fluid of E. coli (pKF2072) and 2-n-butylbenzofuran (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 45 mg of an extract which contained products. The extract was applied onto a silica gel column (Merck 60, Ø 2 cm×30 cm) and then developed with a solvent, hexane:ethyl acetate=10:1, to isolate compound 21 (2-butylbenzo[b]furan-6-ol) (17 mg) and compound 22 (2-butylbenzo[b]furan-5-ol) (5 mg) as a pure substance.

Physical characteristics of compound 21 (2-butylbenzo[b]furan-6-ol)

EI-MS(m/z): 190(M⁺)

¹H-NMR: (500 MHz, CDCl₃): 0.91(t,J=7.3,3H), 1.37(m,2H), 1.67(m,2H), 2.68(m,2H), 4.81(s,1H), 6.24(s,1H), 6.67(dd,J=2.4,7.9,1H), 6.88(s,1H), 7.24(d,J=7.9,1H)

Physical characteristics of compound 22 (2-butylbenzo[b]furan-5-ol)

EI-MS(m/z):190(M⁺)

¹H-NMR:(500 MHz, CDCl₃): 0.91(t,J=7.3,3H), 1.37(m,2H), 1.67(m,2H), 2.67(m,2H), 4.58(s,1H), 6.23(s,1H), 6.76(dd,J=2.0,8.0,1H), 6.84(d,J=2.0,1H), 7.21(d,J=8.0,1H)

5-18. Converted product of 3-n-hexylthiophene (FIG. 7)

To a mixed culture fluid of E. coli (pKF2072) and 3-n-hexylthiophene (700 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 37.5 mg of an extract which contained products. The extract was applied onto a silica gel column (Merck 60, Ø 2 cm×30 cm) and then developed with a solvent, hexane:ethyl acetate=5:1, to isolate compound 23 (4-hexyl-2,3-dihydro-2,3-thiophenediol) (10 mg) as a pure substance.

Physical characteristics of compound 23 (4-hexyl-2,3-dihydro-2,3-thiophenediol)

EI-MS(m/z):202(M⁺)

¹H-NMR:(500 MHz, CDCl₃) 0.88(t,J=7.3,3H), 1.22-1.55(8H), 2.16(t,J=7.3,2H), 4.53(s,1H), 5.54(s,1H), 5.82(s,1H)

### Example 6

Determination of Configurations of Various Converted Products

In order to determine absolute configurations of compounds having a 1,2-dihydroxy-3,5-cyclohexadiene structure, these compounds were first converted into a diester form of (R)-2NMA (methoxy-(2-naphthyl)acetic acid) and (s)-2NMA, and its ¹H-NMR was measured. The chemical shift (δ) of the signal of each ester compound was accurately measured to calculate Δδ (δ Rester—δ Sester). The distribution of this Δδ value was examined to determine its absolute configuration (see FIGS. 6 and 7). The numbers in FIGS. 6 and 7 correspond to the compound numbers in Example 5.

### Example 7

Conversion Experiment Using Recombinant Actinomycetes

Plasmid pKF2072 was double-digested with SacI/SmaI, after which a 4.06 kb fragment containing the modified bphA1::bphA2A3A4 was excised. Then, vector pIJ6021 for actinomycetes (see E. Takano, J. White, C. J. Thompson, M. J. Bibb, Gene, 166, 133-137, 1995) was double-digested with NdeI-HindIII, and then the abovementioned 4.06 kb SacI-HindIII fragment and a synthesized DNA 5′-TATGAGCT-3′ were added. After annealing, treatment with a Klenow fragment enzyme was followed by a ligation reaction. E. coli JM109 strain was transformed, after which the objective plasmid pIJ-2072 was obtained. The pIJ-2072 was designed so that the modified bphA1 gene is located immediately downstream of a powerful actinomycetes promoter P_{tipA} and its ribosome binding site, being followed by the bphA2A3A4 gene. Namely, the 5′ binding site is GAGAAGGGAGCGGACATATGAGCTCATC. The underlined segment is the ribosome binding site, and the modified bphA1 gene starts at ATG from nucleotide 18 to nucleotide 20. This plasmid pIJ-2072 was used to transform actinomycetes Streptomyces lividans TK21 (see D. A. Hopwood, M. J. Bibb, K. F. Chater et al., Genetic Manipulation of Streptomyces: A laboratory Manual, The John Innes Institute, Norwich, 1985).

Cells of the transformant thus obtained were cultured at 30° C. on YEME medium (see Hopwood et al., 1985, supra) supplemented with 5 μg/ml kanamycin up to the second half of the exponential growth phase, after which 5 μg/ml thiostrepton was added to induce the p_{tipA} promoter, and then the cultivation was continued at 30° C. for 24 hours. The resulting cells were washed with the minimal medium (see Hopwood et al., 1985, supra), after which the cells were again suspended in the minimal medium to make the cell concentration to 10 mg (viable cell weight)/ml, and further a substrate such as 2-phenyl quinoline was added at a final concentration of 0.1 mg/ml, and the cultivation was carried out at 30° C. for 2 days. A fatsoluble fraction was extracted from the culture and then HPLC analysis was carried out, according to the methods described in Examples 2 and 3. Results showed that 2-phenylquinoline was converted into its diol derivative (FIG. 6, No. 1) with a yield of almost 100%.

### Example 8

Flavonoid Conversion Reaction

8-1. Flavonoid conversion experiments

Experiments for the conversion of flavone, flavanone, and 6-hydroxyflavanone (FIG. 8) were carried out in the same manner as described in Example 7 using the recombinant actinomycetes (pIJ-2072). More specifically, each of these substrates was added at a final concentration of 1 mM to 1000 ml of the minimal medium containing the cells (10 mg (viable cell weight)/ml), and co-cultivation was carried out at 30° C. for 2 days for the conversion.

8-2. Conversion products of flavone (FIG. 9)

To a mixed culture fluid of recombinant actinomycetes and flavone (1000 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 250 mg of an extract which contained products. The extract was applied onto a silica gel column (Silica Gel 60 (Merck), Ø 2 cm×15 cm) and then developed with a solvent, hexane:ethyl acetate=3:1→1:1, to isolate compound 24 (12 mg) and compound 25 (2.4 mg) as a pure substance.

Compounds 24 and 25 were identified by analyzing various spectrum data (EI-MS, NMR) as follows:

Compound 24: 2′,3′-dihydroxyflavone

Compound 25: 3′-hydroxyflavone

8-3. Converted products of flavanone (FIG. 9)

To a mixed culture fluid of recombinant actinomycetes and flavanone (1000 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 300 mg of an extract which contained products. The extract was applied onto a silica gel column (Silica Gel 60 (Merck), Ø 2 cm×15 cm) and then developed with a solvent, hexane:ethyl acetate=3:1, to isolate compound 26 (13.2 mg), compound 27 (4.4 mg), and compound 28 (4.6 mg) as a pure substance.

Compounds 26, 27 and 28 were identified by analyzing various spectrum data (EI-MS, NMR) as follows:

Compound 26: 2′,3′-dihydroxyflavanone

Compound 27: 2′-hydroxyflavanone

Compound 28: 3′-hydroxyflavanone

Physical characteristics of compound 26 (2′,3′-dihydroxyflavanone)

EI-MS (m/z): 256 (M⁺)

¹H-NMR (500 MHz, DMSO-d6) 2.76 (dd, J=3.0, 16.5, 1H), 3.16 (dd, J=13.0, 16.5, 1H), 5.78 (dd, J=3.0, 13.0, 1H), 6.70 (dd, J=7.9, 7.9), 6.80 (dd, J=1.2, 7.9, 1H), 6.93 (dd, J=1.2, 7.9, 1H), 7.07 (d, J=7.9), 7.08 (dd, J=7.9, 7.9, 1H), 7.57 (ddd, J=1.8, 7.9, 7.9), 7.79 (dd, J=1.8, 7.9, 1H)

8-4. Converted products of 6-hydroxyflavanone (FIG. 9)

To a mixed culture fluid of recombinant actinomycetes and 6-hydroxyflavanone (1000 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 250 mg of an extract which contained products. The extract was applied onto a silica gel column (Silica Gel 60 (Merck), Ø 2 cm×15 cm) and then developed with a solvent, dichloromethane:methanol =50:1, to isolate compound 29 (8.5 mg) and compound 30 (9.0 mg) as a pure substance.

Compounds 29 and 30 were identified by analyzing various spectrum data (EI-MS, NMR) as follows:

Compound 29: 2′,6-dihydroxyflavanone

Compound 30: 3′,6-dihydroxyflavanone

### Example 9

Aromatic Amine Conversion Reaction

9-1. Preparation of Phthalic Acid Imides of Aromatic Amines

An aromatic amine (aromatic compound having a primary amino group) and an equimolar phthalic anhydride were heated at 150° C. for 3 hours in an eggplant-shaped flask. Products were purified on a silica gel column (Silica Gel 60 (Merck), Ø 2 cm×20 cm, hexane:ethyl acetate=5:1). Reactions with all the compounds tested were virtually quantitative.

Further, phthalic acid imide derivatives of amino compounds can be readily converted into their free form by treating with hydrazine hydrate in an alcohol solvent.

9-2. Aromatic Amine Conversion Experiment

Conversion experiments for phthalic acid imide derivatives prepared in 9-1, i.e., a phthalic acid imide derivatives of phenylethylamine [2-(1-phenylethyl)-1,3-isoindolinedione] and a phthalic acid imide derivative of tetrahydronaphthylamine [2-(1,2,3,4-tetrahydro-1-naphthalenyl)-1,3-isoindolinedione] (FIG. 8) was carried out in the same manner as described in Example 7 using the recombinant actinomycetes (pIJ-2072). More specifically, each of these substrates were added at a final concentration of 0.1 mg/ml to 1000 ml of the minimum medium containing the cells (10 mg (viable cell weight)/ml), and co-cultivation was carried out at 30° C. for 2 days for the conversion.

9-3. Converted Product of Phthalic Acid Imide of Phenylethylamine (FIG. 9)

To a mixed culture fluid of recombinant actinomycetes and a phthalic acid imide of phenylethylamine [2-(1-phenylethyl)-1,3-isoindolinedione] (1000 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 350 mg of an extract which contained products. The extract was applied onto a silica gel column (Silica Gel 60 (Merck), Ø 2 cm×10 cm) and then developed with a solvent, hexane:ethyl acetate=3:1, to isolate compound 31 (4.2 mg) as a pure substance.

The compound 31 was identified by analyzing various spectrum data (EI-MS, NMR) as 2-[1-(4-hydroxyphenyl)ethyl]-1,3-isoindolinedione.

Physical characteristics of compound 31 2-[1-(4-hydroxyphenyl)ethyl]-1,3-isoindolinedione

EI-MS (m/z): 267(M⁺)

¹H-NMR(500 MHz, CDCl₃) 1.88 (d, J=7.3, 1H), 5.49 (q, J=7.3, 1H), 6.76 (d, J=8.5, 2H), 7.38 (d, J=8.5, 2H), 7.66 (dd, J=3.1, 5.5, 2H), 7.77 (dd, J=3.1, 5.5, 2H)

9-4. Converted Product of Phthalic Acid Imide of Tetrahydronaphthylamine (FIG. 9)

To a mixed culture fluid of recombinant actinomycetes and a phthalic acid imide of tetrahydronaphthylamine [2-(1,2,3,4-tetrahydro-1-naphthalenyl)-1,3-isoindolinedione)](1000 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml and extracted twice with an equal volume of ethyl acetate. The ethyl acetate layer was concentrated under reduced pressure to obtain 350 mg of an extract which contained products. The extract was applied onto a silica gel column (Silica Gel 60 (Merck), Ø 2 cm×10 cm) and then developed with a solvent, hexane:ethyl acetate=3:1, to isolate compound 32 (5.2 mg) as a pure substance.

The compound 32 was identified by analyzing various spectrum data (EI-MS, NMR) as 2-(4-hydroxy-1,2,3,4-tetrahydro-1-naphthalenyl)-1,3-isoindolinedione.

Physical characteristics of compound 32 2-(4-hydroxy-1,2,3,4-tetrahydro-1-naphthalenyl)-1,3-isoindolinedione

EI-MS (m/z): 293(M⁺)

¹H-NMR (500 MHz, CDCl₃) 1.82 (m, 1H), 2.17 (m, 1H), 2.36-2.44 (2H), 5.01 (dd, J=4.9, 9.2, 1H), 5.56 (dd, J=6.7, 9.8), 6.93 (d, J=7.9, 1H), 7.14 (dd, J=7.9, 7.9, 1H), 7.26 (dd, J=7.9, 7.9, 1H), 7.60 (d, J=7.9, 1H), 7.71 (dd, J=3.0, 5.5, 2H), 7.82 (dd, J=3.0, 5.5, 2H)

### Example 10

Conversion Reaction for Aromatic Carboxylic Acids

10-1. Conversion Experiments for Aromatic Carboxylic Acids

Experiments for the conversion of aromatic carboxylic acids, 1-naphthoic acid and 1-naphthaylacetate (FIG. 8) and 1-naphtoic acid were carried out in the same manner as described in Example 7 using the recombinant actinomycetes (pIJ-2072). More specifically, each of these substrates was added at a final concentration of 0.1 mg/ml to 1000 ml of the minimal medium containing the cells (10 mg (viable cell -weight)/ml), and co-cultivation was carried out at 30° C. for 2 days for the conversion.

10-2. Converted product of 1-naphthoic acid

To a mixed culture fluid of recombinant actinomycetes and 1-naphthoic acid (1000 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml, the pH was adjusted to 3.0 with 1N HCl, and the extraction was carried out twice with an equal volume of ethyl acetate. The ethyl acetate layer was concentrated under reduced pressure to obtain 400 mg of an extract which contained products. The extract was applied onto a silica gel column (Silica Gel 60 (Merck), Ø 2 cm×15 cm) and then developed with a solvent, dichloromethane:methanol =15:1, to isolate compound 33 (5.2 mg) as a pure substance.

Compound 33 was identified as 4-hydroxy-1-naphthoic acid by analyzing various spectrum data (EI-MS, NMR).

Physical characteristics of compound 33 (4-hydroxy-1-naphthoic acid)

EI-MS (m/z): 188 (M⁺)

¹H-NMR (500 MHz, DMSO-d6) 6.90 (d, J=7.9, 1H), 7.49 (dd, J=7.3, 7.3), 7.58 (dd, J=7.3, 9.1), 8.12 (d, J=7.9, 1H), 8.22 (d, J=7.3, 1H), 9.02 (d, J=9.1, 1H)

10-3. Converted products of 1-naphthylacetic acid

To a mixed culture fluid of recombinant actinomycetes and 1-naphthylacetic acid (1000 ml), an equal volume of methanol was added and the admixture was stirred at room temperature for 2 hours. The resultant admixture was centrifuged at 7,000 rpm for 10 minutes to recover a supernatant. This supernatant was concentrated under reduced pressure to 300 ml, the pH was adjusted to 3.0 with 1N HCl , and the extraction was carried out twice with an equal volume of ethyl acetate. The ethyl acetate layer was concentrated under reduced pressure to obtain 380 mg of an extract which contained products. The extract was applied onto a silica gel column (Silica Gel 60 (Merck), Ø 2 cm×15 cm) and then developed with a solvent, dichloromethane:methanol=15:1, to isolate compound 34 (3.6 mg) and compound 35 (2.0 mg) as a pure substance.

Compound 34 and compound 35 were identified by analyzing various spectrum data (El-MS, NMR) as follows:

Compound 34: 4-hydroxy-1-naphthylacetate

Compound 35: 5-hydroxy-1-naphthylacetate

Physical characteristics of compound 34 (4-hydroxy-1-naphthylacetate)

EI-MS (m/z): 202 (M⁺)

¹H-NMR (500 MHz, CDCl₃) 3.96 (s, 2H), 6.72 (d, J=7.9, 1H), 7.18 (d, J=7.9, 1H), 7.45 (dd, J=7.9, 7.9, 1H), 7.51 (dd, 7.9, 8.5), 7.88 (d, J=8.5, 1H), 8.22 (d, J=7.9, 1H)

Physical characteristics of compound 35 (5-hydroxy-1-naphthylacetate)

EI-MS (m/z): 202 (M⁺)

¹H-NMR (500 MHz, CDCl₃) 4.03 (s, 2H), 6.80 (d, J=7.3, 1H), 7.30 (dd, J=7.3, 7.3, 1H), 7.39 (2H), 7.51 (d, J=7.3, 1H), 8.16 (dd, J=2.5, 7.9, 1H)

### SEQUENCE LISTING

<110>kirin beer kabushiki kaisha

<120>a method for producing a heterocyclic compound and an aromatic carboxylic acid having one or more hydroxyl groups, and modified

What is claimed is:

1. A method for producing a hydroxylated heterocyclic compound or a hydroxylated aromatic carboxylic acid comprising the step of reacting an aromatic ring dioxygenase with a heterocyclic compound or an aromatic carboxylic acid to hydroxylate said heterocyclic compound or aromatic carboxylic acid.

2. The method according to claim 1, wherein the aromatic ring dioxygenase is a tetramer consisting of an aromatic ring dioxygenase large subunit (α-subunit), an aromatic ring dioxygenase small subunit (β-subunit), a ferredoxin, and a ferredoxin reductase.

3. The method according to claim 2, wherein the aromatic ring dioxygenase is derived from Pseudomonas pseudoalcaligenes.

4. The method according to claim 2, wherein
the α-subunit consists of the amino acid sequence of SEQ ID NO: 2, or a modified amino acid sequence of SEQ ID NO: 2 having one or more modifications selected from the group consisting of a substitution, a deletion, sn insertion and an addition;
the β-subunit consists of the amino acid sequence of SEQ ID NO: 4 or a modified amino acid sequence of SEQ ID NO: 4 having one or more modifications selected from the group consisting of a substitution, a deletion, an insertion and an addition;
the ferredoxin consists of the amino acid sequence of SEQ ID NO: 6 or a modified amino acid sequence of SEQ ID NO: 6 having one or more modifications selected from the group consisting of a substitution, a deletion, an insertion and an addition;
the ferredoxin reductase consists of the amino acid sequence of SEQ ID NO: 8 or a modified amino acid sequence of SEQ ID NO: 8 having one or more modifications selected from the group consisting of a substitution, a deletion, an insertion and an addition; and
the tetramer consisting of the α-subunit, the β-subunit, the ferredoxin, and the ferredoxin reductase has aromatic ring dioxygenase activity.

5. The method according to claim 2, wherein
the α-subunit consists of the amino acid sequence of SEQ ID NO: 2,
the β-subunit consists of the amino acid sequence of SEQ ID NO: 4,
the ferredoxin consists of the amino acid sequence of SEQ ID NO: 6, and
the ferredoxin reductase consists of the amino acid sequence of SEQ ID NO: 8.

6. The method according to claim 2, wherein
the α-subunit consists of a modified amino acid sequence of SEQ ID NO: 2 which has one or more modifications selected from the group consisting of a substitution, a deletion, an insertion and an addition, and has been modified according to the amino acid sequence of the α-subunit of the biphenyl dioxygenase derived from the strain Burkholderia cepacia LB400;
the β-subunit consists of the amino acid sequence of SEQ ID NO: 4 or a modified amino acid sequence of SEQ ID NO: 4 having one or more modifications selected from the group consisting of a substitution, a deletion, an insertion and an addition;
the ferredoxin consists of the amino acid sequence of SEQ ID NO: 6 or a modified amino acid sequence of SEQ ID NO: 6 having one or more modifications selected from the group consisting of a substitution, a deletion, an insertion and an addition;
the ferredoxin reductase consists of the amino acid sequence of SEQ ID NO: 8 or a modified amino acid sequence of SEQ ID NO: 8 having one or more modifications selected from the group consisting of a substitution, a deletion, an insertion and an addition; and
the tetramer consisting of the α-subunit, the β-subunit, the ferredoxin, and the ferredoxin reductase has aromatic ring dioxygenase activity.

7. The method according to claim 6, wherein the amino acid sequence of the α-subunit derived from the strain Burkholderia cepacia LB400 is the amino acid sequence of SEQ ID NO: 11.

8. The method according to claim 6, wherein the modified amino acid sequence of SEQ ID NO: 2 having modifications is the amino acid sequence of SEQ ID NO: 10.

9. The method according to claim 1, wherein the heterocyclic compound or the aromatic carboxylic acid is hydroxylated by reacting a culture medium, which is obtained by culturing a microorganism transformed to express an aromatic ring dioxygenase gene, with the heterocyclic compound or the aromatic carboxylic acid.

10. The method according to claim 9, wherein the aromatic ring dioxygenase gene consists of a DNA sequence encoding a tetramer consisting of an aromatic ring dioxygenase large subunit (α-subunit), an aromatic ring dioxygenase small subunit (β-subunit), a ferredoxin, and a ferredoxin reductase.

11. The method according to claim 10, wherein the aromatic ring dioxygenase gene is derived from Pseudomonas pseudoalcaligenes.

12. The method according to claim 10, wherein the α-subunit consists of the amino acid sequence of SEQ ID NO: 2, or a modified amino acid sequence of SEQ ID NO: 2 having one or more modifications selected from the group consisting of a substitution, a deletion, an insertion and an addition;
the ′-subunit consists of the amino acid sequence of SEQ ID NO: 4 or a modified amino acid sequence of SEQ ID NO: 4 having one or more modifications selected from the group consisting of a substitution, a deletion, an insertion and an addition;
the ferredoxin consists of the amino acid sequence of SEQ ID NO: 6 or a modified amino acid sequence of SEQ ID NO: 6 having one or more modifications selected from the group consisting of a substitution, a deletion, an insertion and an addition;
the ferredoxin reductase consists of the amino acid sequence of SEQ ID NO: 8 or a modified amino acid sequence of SEQ ID NO: 8 having one or more modifications selected from the group consisting of a substitution, a deletion, an insertion and an addition; and
the tetramer consisting of the α-subunit, the β-subunit, the ferredoxin, and the ferredoxin reductase has aromatic ring dioxygenase activity.

13. The method according to claim 10, wherein
the α-subunit consists of the amino acid sequence of SEQ ID NO: 2,
the β-subunit consists of the amino acid sequence of SEQ ID NO: 4,
the ferredoxin consists of the amino acid sequence of SEQ ID NO: 6, and
the ferredoxin reductase consists of the amino acid sequence of SEQ ID NO: 8.

14. The method according to claim 10 or 13, wherein a DNA sequence encoding the α-subunit is the DNA sequence of SEQ ID NO: 1,
a DNA sequence encoding the β-subunit is the DNA sequence of SEQ ID NO: 3,
a DNA sequence encoding the ferredoxin is the DNA sequence of SEQ ID NO: 5, and
a DNA sequence encoding the ferredoxin reductase is the DNA sequence of SEQ ID NO: 7.

15. The method according to claim 10, wherein
the α-subunit consists of a modified amino acid sequence of SEQ ID NO: 2 which has one or more modifications selected from the group consisting of a substitution, a deletion, an insertion and an addition, and has been modified according to the amino acid sequence of the α-subunit of the biphenyl dioxygenase derived from the strain Burkholderia cepacia LB400;
the β-subunit consists of the amino acid sequence of SEQ ID NO: 4 or a modified amino acid sequence of SEQ ID NO: 4 having one or more modifications selected from the group consisting of a substitution, a deletion, an insertion and an addition;
the ferredoxin consists of the amino acid sequence of SEQ ID NO: 6 or a modified amino acid sequence of SEQ ID NO: 6 having one or more modifications selected from the group consisting of a substitution, a deletion, an insertion and an addition;
the ferredoxin reductase consists of the amino acid sequence of SEQ ID NO: 8 or a modified amino acid sequence of SEQ ID NO: 8 having one or more modifications selected from the group consisting of a substitution, a deletion, an insertion and an addition; and
the tetramer consisting of the α-subunit, the β-subunit, the ferredoxin, and the ferredoxin reductase has aromatic ring dioxygenase activity.

16. The method according to claim 15, wherein the amino acid sequence of the α-subunit derived from the strain Burkholderia cepacia LB400 is the amino acid sequence of SEQ ID NO: 11.

17. The method according to claim 15, wherein the modified amino acid sequence of SEQ ID NO:2 having modifications is the amino acid sequence of SEQ ID NO: 10.

18. The method according to claim 10 or 17, wherein
a DNA sequence encoding the α-subunit is the DNA sequence of SEQ ID NO: 9,
a DNA sequence encoding the β-subunit is the DNA sequence of SEQ ID NO: 3,
a DNA sequence encoding the ferredoxin is the DNA sequence of SEQ ID NO: 5, and
a DNA sequence encoding the ferredoxin reductase is the DNA sequence of SEQ ID NO: 7.

19. The method according to claim 1, wherein the heterocyclic compound is represented by the formula (I):
Het-Alkyl-R¹ (I)
wherein Het is a heterocyclic group, Alkyl is a bond or an optionally-branched alkylene chain having 1 to 4 carbon atoms, and R¹ is an unsubstituted phenyl group.

20. The method according to claim 1, wherein the hydroxylated heterocyclic compound is represented by the formula (I′):
Het-Alkyl-R^{1′} (I′)
wherein Het and Alkyl are the same as defined in claim 19, and R^{1′} is any one of the following groups:

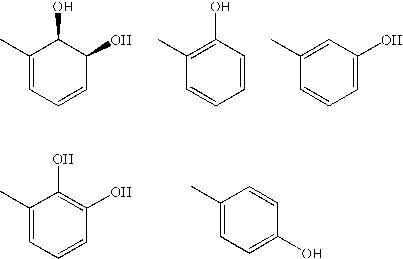

21. The method according to claim 19 or 20, wherein Het is quinoline, indole, indanone, benzothiazole, benzoxazole, pyridine, 3-methylpyridine, pyrimidine, pyrrole, pyrazole, 3-methylpyrazole, imidazole, isothiazole, benzofuran, thiophene, chromone (4H-chromene-4-on), chroman-4-on, 6-hydroxy-chroman-4-on, or phthalimide.

22. The method according to claim 1, wherein the heterocyclic compound is represented by the formula (II):
Het-Alkyl-R² (II)
wherein Het is a heterocyclic group, Alkyl is a bond or an optionally-branched alkylene chain having 1 to 4 carbon atoms, and R² is a phenyl group substituted with a C₁₋₄ alkyl group or a hydroxyl group.

23. The method according to claim 22, wherein Het is benzoxazole or pyridine, and R² is 2-hydroxyphenyl or 4-methylphenyl.

24. The method claimed in claim 1, wherein the hydroxylated heterocyclic compound is represented by the formula (II′):
Het′-Alkyl-R² (II′)
wherein R² and Alkyl are the same as defined in claim 22, and Het′ is a heterocyclic group substituted with 1 or 2 hydroxyl groups.

25. The method according to claim 24, wherein Het′ is 4,5-dihydroxy-4,5-dihydrobenzoxazole or3-hydroxypyridine.

26. The method according to claim 1, wherein the heterocyclic compound is represented by the formula (III):
Het-Alkyl-H (III)
wherein Het is a heterocyclic group, Alkyl is an optionally-branched alkylene chain having 1 to 8 carbon atoms.

27. The method according to claim 26, wherein Het is benzofuran or thiophene.

28. The method according to claim 1, wherein the hydroxylated heterocyclic compound is represented by the formula (III′):
Het′-Alkyl-H (III′)
wherein Het′ is a heterocyclic group substituted with 1 or 2 hydroxyl groups and Alkyl is the same as defined in claim 26.

29. The method according to claim 28, wherein Het′ is 3-hydroxybenzofuran, 4-hydroxybenzofuran, or 2,3-dihydroxy-2,3-dihydrothiophene.

30. The method according to claim 1, wherein the heterocyclic compound and the hydroxylated heterocyclic compound are selected from the following combinations:

| Heterocyclic compound | Hydroxylated heterocyclic compound |
|---|---|
| 2-Phenyl quinoline | 3-(2-Quinolyl)-3,5-cyclohexadiene- |
| | 1,2-diol |
| 2-Phenyl indole | 3-(1H-2-Indolyl)-3,5-cyclohexadiene- |
| | 1,2-diol |
| 2-Phenyl indole | 2-(1H-2-Indolyl)phenol |
| 2-Phenyl indole | 2-Phenyl-1H-5-indolol |
| 3-Phenyl-1-indanone | 3-(5,6-Dihydroxy-1,3- |
| | cyclohexadienyl)-1-indanone |
| 2-Phenyl | 3-(1,3-Benzothiazole-2-yl)-3,5- |
| benzothiazole | cyclohexadiene-1,2-diol |
| 2-Phenyl benzoxazole | 3-(1,3-Benzoxazole-2-yl)-3,5- |
| | cyclohexadiene-1,2-diol |
| 2-Phenyl pyridine | 3-(2-Pyridyl)-3,5-cyclohexadiene- |
| | 1,2-diol |
| 3-Metyl-2-phenyl | 3-(3-Methylpyrido-2-yl)-3,5- |
| pyridine | cyclohexadiene-1,2-diol |
| 4-Phenyl pyrimidine | 3-(4-Pyrimidinyl)-3,5- |
| | cyclohexadiene-1,2-diol |
| 1-Phenyl pyrrole | 3-(1H-1-Pyrrolyl)-3,5- |
| | cyclohexadiene-1,2-diol |
| 1-Phenyl pyrazole | 4-Hydroxy-1-phenylpyrazole |
| 3-Metyl-1-phenyl | 3-(3-Methylpyrazole-1-yl)-3,5- |
| pyrazole | cyclohexadiene-1,2-diol |
| 3-Metyl-1-phenyl | 2-(3-Methylpyrazole-1-yl)-phenol |
| pyrazole | |
| 2-Benzyl pyridine | 3-(2-Pyridylmethyl)-3,5- |
| | cyclohexadiene-1,2-diol |
| 1-Benzyl imidazole | 3-(1H-1-Imidazolylmethyl)-3,5- |
| | cyclohexadiene-1,2-diol |
| 4-Benzyl isothiazole | 3-(4-Isothiazolylmethyl)-3,5- |
| | cyclohexadiene-1,2-diol |
| 4-Benzyl isothiazole | 2-(4-Isothiazolylmethyl)phenol |
| 2-(2-Hydroxyphenyl)- | 2-(2-Hydroxyphenyl)-4,5-dihydro-1,3- |
| benzoxazole | benzoxazole-4,5-diol |
| 2-(p-Tolyl)pyridine | 2-(4-Methylphenyl)-3-pyridiol |
| 2-n-Butylbenzofuran | 2-Butylbenzo[b]furan-6-ol |
| 2-n-Butylbenzofuran | 2-Butylbenzo[b]furan-5-ol |
| 3-n-Hexyl thiophene | 4-Hexyl-2,3-dihydro-2,3- |
| | thiophenediol |
| Flavone | 2′,3′-Dihydroxyflavone |
| Flavone | 3′-Hydoxyflavone |
| Flavanone | 2′,3′-Dihydroxyflavanone |
| Flavanone | 2′-Hydoxyflavanone |
| Flavanone | 3′-Hydoxyflavanone |
| 6-Hydroxyflavanone | 2′,6-Dihydroxyflavanone |
| 6-Hydroxyflavanone | 3′,6-Dihydroxyflavanone |
| 2-(1-Phenylethyl)- | 2-[1-(4-Hydroxyphenyl)ethyl]-1,3- |
| 1,3-isoindolinedione | isoindolinedione |
| 2-(1,2,3,4- | 2-(4-Hydroxy-1,2,3,4-tetrahydro-1- |
| Tetrahydro-1- | naphthalenyl)- |
| naphthalenyl)-1,3- | 1,3-isoindolinedione |
| isoindolinedione | |

31. The method according to claim 1, wherein the heterocyclic compound is a flavonoid.

32. The method according to claim 31, wherein the flavonoid is flavone, flavanone, or 6-hydroxyflavanone.

33. The method according to claim 31, wherein hydroxylated flavonoid is a 2′,3′-dihydroxy derivative, a 2′-hydroxy derivative or a 3′-hydroxy derivative.

34. The method according to claim 33, wherein the hydroxylated flavonoid is 2′,3′-dihydroxyflavone, 3′-hydroxyflavone, 2′,3′-dihydroxyflavanone, 2′-hydroxyflavanone, 3′-hydroxyflavanone, 2′,6-dihydroxyflavanone, or 3′,6-dihydroxyflavanone.

35. The method according to any one of claims 31 to 34, wherein the flavonoid and the hydroxylated flavonoid are selected from the following combinations:

| Flavonoid | Hydroxylated flavonoid |
|---|---|
| Flavone | 2′,3′-Dihydroxyflavone |
| Flavone | 3′-Hydoxyflavone |
| Flavanone | 2′,3′-Dihydroxyflavanone |
| Flavanone | 2′-Hydoxyflavanone |
| Flavanone | 3′-Hydoxyflavanone |
| 6-Hydroxyflavanone | 2′,6-Dihydroxyflavanone |
| 6-Hydroxyflavanone | 3′,6-Dihydroxyflavanone |

36. The method according to claim 1, wherein the heterocyclic compound is a phthalimide derivative having an aromatic ring.

37. The method according to claim 36, wherein the phthalimide derivative having an aromatic ring is 2-(1-phenylethyl)-1,3-isoindolinedione or 2-(1,2,3,4-tetrahydro-1-naphthalenyl)-1,3-isoindolinedione.

38. The method according to claim 36, wherein the hydroxylated phthalimide derivative having an aromatic ring is a hydroxylated phthalimide derivative of which the aromatic ring or the benzyl group is hydroxylated.

39. The method according to claim 38, wherein the hydroxylated phthalimide derivative having an aromatic ring is 2-[1-(4-hydroxyphenyl)ethyl]-1,3-isoindolinedione or 2-(4-hydroxy-1,2,3,4-tetrahydro-1-naphthalenyl)-1,3-isoindolinedione.

40. The method according to any one of claims 36 to 39, wherein the phthalimide derivative having an aromatic ring and the hydroxylated phthalimide derivative having an aromatic ring are selected from the following combinations:

| Phthalimide derivative | Hydroxylated phthalimide derivative |
|---|---|
| having an aromatic ring | having an aromatic ring |
| 2-(1-Phenylethyl)-1,3- | 2-[1-(4-Hydroxyphenyl)ethyl]-1,3- |
| isoindolinedione | isoindolinedione |
| 2-(1,2,3,4-Tetrahydro-1- | 2-(4-Hydroxy-1,2,3,4-tetrahydro-1- |
| naphthalenyl)-1,3- | naphthalenyl)- |
| isoindolinedione | 1,3-isoindolinedione |

41. The method according to claim 1, wherein the aromatic carboxylic acid is represented by the formula (IV):
R³-Alkyl-COOR⁴ (IV)
wherein R³ is an unsubstituted carbon ring group, Alkyl is a bond or an optionally-branched alkylene chain having 1 to 4 carbon atoms, and R⁴is a hydrogen atom or a protecting group for a carboxyl group.

42. The method according to claim 41, wherein R³ is naphthalene.

43. The method according to claim 41, wherein the compound of the formula (IV) is 1-naphtoic acid or 1-naphthylacetic acid.

44. The method according to claim 1, wherein the hydroxylated aromatic carboxylic acid is represented by the formula (IV′):
R^{3′}-Alkyl-COOR⁴ (IV′)
wherein Alkyl and R⁴ are the same as defined above, and R^{3′} is a carbon cyclic group substituted with 1 or 2 hydroxyl groups.

45. The method according to claim 44, wherein R^{3′} is naphthalene substituted with 1 or 2 hydroxyl groups.

46. The method according to claim 44, wherein the compound of the formula (IV′) is 4-hydroxy-1-naphthoic acid, 4-hydroxy-1-naphthylacetic acid, or 5-hydroxy-1-naphthylacetic acid.

47. The method according to claim 1, wherein the aromatic carboxylic acid and the hydroxylated aromatic carboxylic acid are selected from the following combinations:

| Aromatic carboxylic | Hydroxylated aromatic |
|---|---|
| acid | carboxylic acid |
| 1-Naphthoic acid | 4-Hydroxy-1-naphthoic acid |
| 1-Naphthylacetic acid | 4-Hydroxy-1-naphthylacetic acid |
| 1-Naphthylacetic acid | 5-Hydroxy-1-naphthylacetic acid |

48. The method according to claim 1, wherein the microorganism is Escherichia coli, actinomycetes, or yeast.

49. An aromatic ring dioxygenase comprising
an α-subunit consisting of a modified amino acid sequence of SEQ ID NO: 2 which has one or more modifications selected from the group consisting of a substitution, a deletion, an insertion and an addition, and has been modified according to the amino acid sequence of the α-subunit of the biphenyl dioxygenase derived from the strain Burkholderia cepacia LB400;
a β-subunit consisting of the amino acid sequence of SEQ ID NO: 4 or a modified amino acid sequence of SEQ ID NO: 4 having one or more modifications selected from the group consisting of a substitution, a deletion, an insertion and an addition;
a ferredoxin consisting of the amino acid sequence of SEQ ID NO: 6 or a modified amino acid sequence of SEQ ID NO: 6 having one or more modifications selected from the group consisting of a substitution, a deletion, an insertion and an addition; and
a ferredoxin reductase consisting of the amino acid sequence of SEQ ID NO: 8 or a modified amino acid sequence of SEQ ID NO: 8 having one or more modifications selected from the group consisting of a substitution, a deletion, an insertion and an addition.

50. The aromatic ring dioxygenase according to claim 49, wherein the α-subunit consists of the amino acid sequence of SEQ ID NO: 10.

51. A polynucleotide encoding the aromatic ring dioxygenase claimed in claim 49 or 50.

52. A protein consisting of the amino acid sequence of SEQ ID NO: 10.

53. A polynucleotide encoding the protein claimed in claim 52.

54. A method of introducing a hydroxyl group into a heterocyclic compound or an aromatic carboxylic acid comprising the step of reacting an aromatic ring dioxygenase with the heterocyclic compound or the aromatic carboxylic acid.

55. The method according to claim 54, wherein the aromatic ring dioxygenase is that claimed in claim 49 or 50.

56. A composition for hydroxylating a heterocyclic compound or an aromatic carboxylic acid comprising an aromatic ring dioxygenase.

57. The composition according to claim 56, wherein the aromatic ring dioxygenase is that claimed in claim 49 or 50.


SEQUENCE LISTING (14 sequences)

SEQ ID NO: 1 - DNA, 1377 bp, Pseudomonas pseudoalcaligenes
  CDS (1)..(1374)
atgagctcagcaatcaaagaagtgcagggagcccctgtgaagtgggttaccaattggacgccggaggcgatccgggggttggtcgatcaggaaaaagggctgcttgatccacgcatctacgccgatcagagtctttatgagctggagcttgagcgggtttttggtcgctcttggctgttacttgggcacgagagtcatgtgcctgaaaccggggacttcctggccacttacatgggcgaagatccggtggttatggtgcgacagaaagacaagagcatcaaggtgttcctgaaccagtgccggcaccgcggcatgcgtatctgccgctcggacgccggcaacgccaaggctttcacctgcagctatcacggctgggcctacgacatcgccggcaagctggtgaacgtgccgttcgagaaggaagccttttgcgacaagaaagaaggcgactgcggctttgacaaggccgaatggggcccgctccaggcacgcgtggcaacctacaagggcctggtctttgccaactgggatgtgcaggcgccagacctggagacctacctcggtgacgcccgcccctatatggacgtcatgctggatcgcacgccggccgggactgtggccatcggcggcatgcagaagtgggtgattccgtgcaactggaagtttgccgccgagcagttctgcagtgacatgtaccacgccggcaccatgtcgcacctgtccggcatcctggcgggcatgccgccggaaatggacctgtcgcatgcacaggtgcccaccaagggcaaccagttccgggccggctggggcgggcacggctcgggctggttcgtcgacgagccgggcatgctcatggcggtgatggggcccaaggtcacccagtactggaccgaaggtccggctgccgacctggcagaacagcgactgggccacaccatgccggttcgacgcatgttcggccagcacatgagcgtcttcccgacctgctcgttcctcccggccatcaacaccatccggacctggcacccgcgcggccccaacgaaatcgaagtgtgggccttcaccttggtcgatgccgatgccccggccgagatcaaggaagaatatcgccggcacaacatccgcaccttctccgcaggcggcgtgtttgagcaggacgatggcgagaactgggtggagatccagaaggggctacgtgggtacaaggccaagagccagccgctcaatgcccagatgggcctgggtcggtcgcagaccggtcaccctgattttcctggcaacgtcggctacgtctacgccgaagaagcggcgcggggtatgtatcaccactggatgcgcatgatgtccgagcccagctgggccacgctcaagccctga

SEQ ID NO: 2 - protein, 458 aa, Pseudomonas pseudoalcaligenes
MSSAIKEVQGAPVKWVTNWTPEAIRGLVDQEKGLLDPRIYADQSLYELELERVFGRSWLLLGHESHVPETGDFLATYMGEDPVVMVRQKDKSIKVFLNQCRHRGMRICRSDAGNAKAFTCSYHGWAYDIAGKLVNVPFEKEAFCDKKEGDCGFDKAEWGPLQARVATYKGLVFANWDVQAPDLETYLGDARPYMDVMLDRTPAGTVAIGGMQKWVIPCNWKFAAEQFCSDMYHAGTMSHLSGILAGMPPEMDLSHAQVPTKGNQFRAGWGGHGSGWFVDEPGMLMAVMGPKVTQYWTEGPAADLAEQRLGHTMPVRRMFGQHMSVFPTCSFLPAINTIRTWHPRGPNEIEVWAFTLVDADAPAEIKEEYRRHNIRTFSAGGVFEQDDGENWVEIQKGLRGYKAKSQPLNAQMGLGRSQTGHPDFPGNVGYVYAEEAARGMYHHWMRMMSEPSWATLKP

SEQ ID NO: 3 - DNA, 642 bp, Pseudomonas alcaligenes
  CDS (1)..(639)
atggtgggctggacgtgcatgtgcagacggcgcgccgaggttccgtcccctgatatttacttggagataactgttatgacaaatccatccccgcattttttcaaaacatttgaatggccaagcaaggcggctggccttgagttgcagaacgagatcgagcagttctactaccgcgaagcgcagttgcttgaccaccgggcctacgaggcctggtttgccctgctggacaaagatatccactacttcatgccgctgcgcaccaatcgcatgatccgggagggcgagctggaatattccggcgaccaggatgttgcccatttcgatgaaacccatgaaaccatgtacgggcgcatccgcaaggtgacctcggacgtgggctgggcggagaacccgccttcccgcacgcgccacctggtctccaacgtcatcgtcaaggagacggccacgccggataccttcgaggtcaattccgcattcatcctgtaccgcaatcggcttgagcgccaggtcgacatcttcgcgggcgaacgccgggacgtgctgcgccgcgccgacaacaaccttggtttcagcatcgccaagcgcaccatcctgctcgacgccagtaccttgctgtcgaacaacctgagcatgttcttctag

SEQ ID NO: 4 - protein, 213 aa, Pseudomonas alcaligenes
MVGWTCMCRRRAEVPSPDIYLEITVMTNPSPHFFKTFEWPSKAAGLELQNEIEQFYYREAQLLDHRAYEAWFALLDKDIHYFMPLRTNRMIREGELEYSGDQDVAHFDETHETMYGRIRKVTSDVGWAENPPSRTRHLVSNVIVKETATPDTFEVNSAFILYRNRLERQVDIFAGERRDVLRRADNNLGFSIAKRTILLDASTLLSNNLSMFF

SEQ ID NO: 5 - DNA, 330 bp, Pseudomonas alcaligenes
  CDS (1)..(327)
atgaaatttaccagagtttgtgatcgaagagatgtgcccgaaggcgaagccctgaaggtcgaaagtggaggcacctccgtcgcgattttcaatgtggatggcgagctgttcgcaacacaggaccgctgcacccacggcgactggtccctgtccgatggcggctatcttgaaggtgacgtggtggaatgctcactgcacatggggaagttttgcgttcgcacgggcaaggtcaaatcaccgccgccctgtgaggcactgaagatatttccgatccgcatcgaagacaatgacgtgctggtcgacttcgaagccgggtatctggcgccatga

SEQ ID NO: 6 - protein, 109 aa, Pseudomonas alcaligenes
MKFTRVCDRRDVPEGEALKVESGGTSVAIFNVDGELFATQDRCTHGDWSLSDGGYLEGDVVECSLHMGKFCVRTGKVKSPPPCEALKIFPIRIEDNDVLVDFEAGYLAP

SEQ ID NO: 7 - DNA, 1227 bp, Pseudomonas alcaligenes
  CDS (1)..(1224)
atgatcgacaccatcgccatcatcggcgccggcctggccggttcgacggctgcgcgcgcactgcgcgcccagggatacgaggggcgcatccacctgctcggggatgagtcgcatcaggcctatgaccggaccacgctgtccaagacggtgctggcgggcgagcagcccgagccgcctgcaatcctggacagcgcctggtacgcatcggcccatgtggatgtccagctcgggcgacgggtgagttgcctggatctggccaaccgccagattcagtttgaatcgggcgccccgctggcctacgaccggctgctgctggccaccggcgcgcgcgcccggcgcatggcgattcggggtggcgacctggcaggcatccataccttgcgagacctcgccgacagccaggcgctgcggcaggcgctgcaaccgggccagtcgctggtcatcgtcggcggaggcctgatcggttgcgaggtggcgaccaccgcccgcaagctgagtgtccatgtcacgattctggaagccggcgacgagttgctggtgcgcgtgctgggtcaccggaccggggcatggtgtcgggccgaactggaacgcatgggtgtccgcgtggagcgcaatgcacaggccgcgcgcttcgaaggccaggggcaggtgcgcgccgtgatctgcgccgacgggcgccgggtgcccgccgatgtggtcttggtcagcattggcgccgagccggcggacgagctggcccgtgccgctggcatcgcctgcgcgcgcggcgtgctggtcgacgccaccggcgccacctcgtgtccagaggtgttcgccgccggtgacgtcgccgcctggccgctgcgtcaagggggccagcgctcgctggagacctacctgaacagccagatggaggccgaaatcgcggccagcgccatgttgagtcagcccgtgccggcgccccaggtgccgacctcgtggacggagattgcaggccaccgcatccagatgattggcgatgccgaagggcccggcgagatcgtcgtacgcggcgacgcccagagcggccagccaatcgtgttgctcaggctgcttgatggctgcgtcgaggccgcgacggcgatcaatgccaccagggaattttctgtggcgacccgactggtcggcacccgggtttctgtttccgccgagcaactgcaggacgtcggctcgaacctgcgggatttactcaaagccaaaccgaattga

SEQ ID NO: 8 - protein, 408 aa, Pseudomonas alcaligenes
MIDTIAIIGAGLAGSTAARALRAQGYEGRIHLLGDESHQAYDRTTLSKTVLAGEQPEPPAILDSAWYASAHVDVQLGRRVSCLDLANRQIQFESGAPLAYDRLLLATGARARRMAIRGGDLAGIHTLRDLADSQALRQALQPGQSLVIVGGGLIGCEVATTARKLSVHVTILEAGDELLVRVLGHRTGAWCRAELERMGVRVERNAQAARFEGQGQVRAVICADGRRVPADVVLVSIGAEPADELARAAGIACARGVLVDATGATSCPEVFAAGDVAAWPLRQGGQRSLETYLNSQMEAEIAASAMLSQPVPAPQVPTSWTEIAGHRIQMIGDAEGPGEIVVRGDAQSGQPIVLLRLLDGCVEAATAINATREFSVATRLVGTRVSVSAEQLQDVGSNLRDLLKAKPN

SEQ ID NO: 9 - DNA, 1377 bp, Artificial Sequence
  Description of Artificial Sequence Modified dioxygenase gene derived from P. pseudoalcaligenes
atgagctcagcaatcaaagaagtgcagggagcccctgtgaagtgggttaccaattggacgccggaggcgatccgggggttggtcgatcaggaaaaagggctgcttgatccacgcatctacgccgatcagagtctttatgagctggagcttgagcgggtttttggtcgctcttggctgttacttgggcacgagagtcatgtgcctgaaaccggggacttcctggccacttacatgggcgaagatccggtggttatggtgcgacagaaagacaagagcatcaaggtgttcctgaaccagtgccggcaccgcggcatgcgtatctgccgctcggacgccggcaacgccaaggctttcacctgcagctatcacggctgggcctacgacatcgccggcaagctggtgaacgtgccgttcgagaaggaagccttttgcgacaagaaagaaggcgactgcggctttgacaaggccgaatggggcccgctccaggcacgcgtggcaacctacaagggcctggtctttgccaactgggatgtgcaggcgccagacctggagacctacctcggtgacgcccgcccctatatggacgtcatgctggatcgcacgccggccgggactgtggccatcggcggcatgcagaagtgggtgattccgtgcaactggaagtttgccgccgagcagttctgcagtgacatgtaccacgccggcaccatgtcgcacctgtccggcatcctggcgggcatgccgccggaaatggacctctcccaggcgcagatacccaccaagggcaatcagttccgggccgcttggggcgggcacggctcgggctggtatgtcgacgagccgggcatgctcatggcggtgatggggcccaaggtcacccagtactggaccgaaggtccggctgccgacctggcagaacagcgactgggccacaccatgccggttcgacgcatgttcggccagcacatgagcgtcttcccgacctgctcgttcctcccggccatcaacaccatccggacctggcacccgcgcggccccaacgaaatcgaagtgtgggccttcaccttggtcgatgccgatgccccggccgagatcaaggaagaatatcgccggcacaacatccgcaccttctccgcaggcggcgtgtttgagcaggacgatggcgagaactgggtggagatccagaaggggctacgtgggtacaaggccaagagccagccgctcaatgcccagatgggcctgggtcggtcgcagaccggtcaccctgattttcctggcaacgtcggctacgtctacgccgaagaagcggcgcggggtatgtatcaccactggatgcgcatgatgtccgagcccagctgggccacgctcaagccctga

SEQ ID NO: 10 - protein, 458 aa, Artificial Sequence
  Description of Artificial Sequence Modified dioxygenase gene derived from P. pseudoalcaligenes
MSSAIKEVQGAPVKWVTNWTPEAIRGLVDQEKGLLDPRIYADQSLYELELERVFGRSWLLLGHESHVPETGDFLATYMGEDPVVMVRQKDKSIKVFLNQCRHRGMRICRSDAGNAKAFTCSYHGWAYDIAGKLVNVPFEKEAFCDKKEGDCGFDKAEWGPLQARVATYKGLVFANWDVQAPDLETYLGDARPYMDVMLDRTPAGTVAIGGMQKWVIPCNWKFAAEQFCSDMYHAGTMSHLSGILAGMPPEMDLSQAQIPTKGNQFRAAWGGHGSGWYVDEPGMLMAVMGPKVTQYWTEGPAADLAEQRLGHTMPVRRMFGQHMSVFPTCSFLPAINTIRTWHPRGPNEIEVWAFTLVDADAPAEIKEEYRRHNIRTFSAGGVFEQDDGENWVEIQKGLRGYKAKSQPLNAQMGLGRSQTGHPDFPGNVGYVYAEEAARGMYHHWMRMMSEPSWATLKP

SEQ ID NO: 11 - protein, 459 aa, Burkholderia cepacia
MSSAIKEVQGAPVKWVTNWTPEAIRGLVDQEKGLLDPRIYADQSLYELELERVFGRSWLLLGHESHVPETGDFLATYMGEDPVVMVRQKDKSIKVFLNQCRHRGMRICRSDAGNAKAFTCSYHGWAYDIAGKLVNVPFEKEAFCDKKEGDCGFDKAEWGPLQARVATYKGLVFANWDVQAPDLETYLGDARPYMDVMLDRTPAGTVAIGGMQKWVIPCNWKFAAEQFCSDMYHAGTTTHLSGILAGIPPEMDLSHAQVPTKGNQFRAGWGGHGSGWFVDEPGSLLAVMGPKVTQYWTEGPAAELAEQRLGHTGMPVRRMVGQHMTIFPTCSFLPTFNNIRIWHPRGPNEIEVWAFTLVDADAPAEIKEEYRRHNIRNFSAGGVFEQDDGENWVEIQKGLRGYKAKSQPLNAQMGLGRSQTGHPDFPGNVGYVYAEEAARGMYHHWMRMMSEPSWATLKP

SEQ ID NO: 12 - DNA, 35 bp, Artificial Sequence
  Description of Artificial Sequence Primer
ccgaattcaaggagacgttgaatcatgagctcagc

SEQ ID NO: 13 - DNA, 25 bp, Artificial Sequence
  Description of Artificial Sequence Primer
ttgaattcttccggttgacagatct

SEQ ID NO: 14 - DNA, 28 bp, Artificial Sequence
  Description of Artificial Sequence Synthetic oligonucleotide
gagaagggagcggacatatgagctcatc